(19) United States
(12) Patent Application Publication
(10) Pub. No.: US 2021/0401683 A1
(43) Pub. Date: Dec. 30, 2021

(54) METHOD FOR TREATING KERATIN MATERIALS USING AN ACRYLIC ANHYDRIDE POLYMER IN OILY DISPERSION AND AN AMINE COMPOUND

(71) Applicant: L'OREAL, Paris (FR)

(72) Inventors: Julien PORTAL, Aulnay-Sous-Bois (FR); Simon TAUPIN, Aulnay-Sous-Bois (FR)

(21) Appl. No.: 17/284,179

(22) PCT Filed: Oct. 11, 2019
(86) PCT No.: PCT/EP2019/077630

§ 371 Date: Apr. 9, 2021

(30) Foreign Application Priority Data
Oct. 11, 2018 (FR) 1859443

(51) Int. Cl. A61K 8/06; A61K 8/81; A61K 8/41; A61K 8/898; A61Q 1/06

(52) U.S. Cl. CPC A61K 8/064; A61K 8/8152; A61K 8/41; A61K 2800/43; A61Q 1/06; A61K 2800/52; A61K 2800/60; A61K 8/898

(57) ABSTRACT

The present invention relates to a process for treating keratin materials, preferably human skin, in particular the lips, preferably in at least two steps involving, in a first stage, the application to said materials of an oily dispersion (A) comprising i) particles of copolymers of alkyl acrylates and of anhydride acrylics, ii) stabilizers, iii) one or more hydrocarbon-based oils; and then, in a second stage, the application to said materials of a composition (B) comprising iv) one or more amine compounds, it being understood that the process of the invention uses v) one or more cosmetic active agents chosen from a) pigments, b) active agents for caring for the skin, and c) UV-screening agents and also d) mixtures thereof; ingredients a) to d) possibly being found in composition (A), and/or in composition (B), and/or in another composition (C).

The process of the invention makes it possible to obtain a treatment of said keratin materials that is notably resistant to sweat, to shower gels, to water and to fatty substances, in particular plant or animal food oils, and more particularly plant oils such as olive oil, sunflower oil, walnut oil, hazelnut oil, etc.

The keratin material treatment process of the present patent application is also suitable for making up the skin or the lips, such as foundations and lipsticks.

The present invention relates to a process for treating keratin materials, preferably human skin, in particular the lips and preferably in at least two steps involving, in a first stage, the application to said materials of an oily dispersion (A) comprising i) particles of copolymers of alkyl acrylates and of anhydride acrylics, ii) stabilizers, iii) one or more hydrocarbon-based oils; and then, in a second stage, the application to said materials of a composition (B) comprising iv) one or more amine compounds, it being understood that the process of the invention uses v) one or more cosmetic active agents chosen from a) pigments, b) active agents for caring for keratin materials, preferably the skin, and c) UV-screening agents and also d) mixtures thereof; ingredients a) to d) possibly being found in composition (A), and/or in composition (B), and/or in another composition (C).

During the aging process, various signs appear on the skin which are very characteristic of this aging, reflected notably by a change in the skin structure and functions. The main clinical signs of skin aging are notably the appearance of fine lines and deep wrinkles, which increase with age.

It is known practice to treat these signs of aging using cosmetic or dermatological compositions containing active agents that are capable of combating aging, such as α-hydroxy acids, β-hydroxy acids and retinoids. These active agents act on wrinkles by eliminating dead skin cells and by accelerating the cell renewal process. However, these active agents have the drawback of being effective for the treatment of wrinkles only after a certain application time. Now, it is increasingly sought to obtain an immediate effect of the active agents used, rapidly resulting in smoothing-out of wrinkles and fine lines and in the disappearance of fatigue marks.

Cosmetic products often require the use of a film-forming polymer to obtain a deposit of the product on keratin materials that has good cosmetic properties. In particular, it is necessary for the film-forming deposit to have good resistance, in particular for the deposit not to transfer during contact with the fingers or clothing, and also good resistance on contact with water, notably rain or during showering or perspiration, and also food fats, notably dietary oils.

It is known practice to use dispersions of polymer particles of nanometric size, in organic media such as hydrocarbon-based oils. Polymers are notably used as film-forming agents in makeup products such as mascaras, eyeliners, eyeshadows or lipsticks. EP-A-749 747 describes in the examples dispersions in hydrocarbon-based oils (liquid paraffin, isododecane) of acrylic polymers stabilized with polystyrene/copoly(ethylene-propylene) diblock copolymers. The film obtained after application of the dispersion to the skin is sparingly glossy. FR 1362795 also describes the use of dispersions of surface-stabilized polymer particles containing hydrocarbon-based oils for making up the lips and eyelashes. WO-A-2010/046229 describes dispersions in isododecane of acrylic polymers stabilized with stabilizing polymers. In the prepared by reversible chain-transfer controlled radical polymerization. FR 1 362 795 describes the use of dispersions of surface-stabilized polymer particles containing hydrocarbon-based oils for making up the lips and the eyelashes. In addition, the glossy effect notably on the lips is not always satisfactory.

Thus, the aim of the present invention is to provide a method for treating keratin materials, in particular the skin, preferably human skin and more preferentially facial skin, which has good resistance with respect to extemal attacking factors, and over time, of the action of the cosmetic active agent(s) such as the tensioning, care or ultraviolet (UV) protection effect, but also good persistence of makeup such as lip makeup.

The technical problem has been solved by the process of the invention, which is a process for treating keratin materials, in particular the skin, preferably human skin and more preferentially facial skin such as the lips, comprising:
1) the application to said materials of an oily dispersion (A), which is preferably anhydrous, comprising:
   i) one or more particles consisting of one or more ethylenic copolymers:
      a) of (C₁-C₄)alkyl (C₁-C₄)(alkyl)acrylate, preferably (C₁-C₄)alkyl (meth)acrylate; and
      b) of ethylenically unsaturated anhydride compound, preferably maleic anhydride; and
   ii) one or more stabilizers consisting of ethylenic polymers chosen from:
      c) polymers of (C₃-C₁₂)cycloalkyl (C₁-C₆)(alkyl)acrylate monomers; and
      d) copolymers of (C₃-C₁₂)cycloalkyl (C₁-C₆)(alkyl)acrylate and (C₁-C₄)alkyl (C₁-C₄)(alkyl)acrylate; and
   iii) one or more hydrocarbon-based oils;
2) the application to said materials of a composition (B), which is preferably anhydrous, comprising:
   iv) one or more amine compounds chosen from:
      e) polyamine compounds bearing several primary amine and/or secondary amine groups, and
      f) amino alkoxysilanes,

it being understood that:
the process of the invention uses v) one or more cosmetic active agents chosen from a) pigments, b) active agents for caring for keratin materials, preferably the skin, and c) UV-screening agents, and also d) mixtures thereof, ingredients a) to d) possibly being found in composition (A), and/or in composition (B), and/or in another composition (C); and
compositions (A), (B) and (C) may be applied together or separately.

More preferentially, the process of the invention is a process for treating keratin materials, in particular the skin, preferably human skin and more preferentially facial skin such as the lips, comprising:
1) the application to said materials of an oily dispersion (A), which is preferably anhydrous, comprising:
   i) one or more particles consisting of one or more ethylenic copolymers:
      a) of (C₁-C₄)alkyl (C₁-C₄)(alkyl)acrylate, preferably (C₁-C₄)alkyl (meth)acrylate; and
      b) of ethylenically unsaturated anhydride compound, preferably maleic anhydride; and
   ii) one or more stabilizers consisting of ethylenic polymers chosen from:
      C) polymers of (C₃-C₁₂)cycloalkyl (C₁-C₆)(alkyl)acrylate monomers; and
      d) copolymers of (C₃-C₁₂)cycloalkyl (C₁-C₆)(alkyl)acrylate and (C₁-C₄)alkyl (C₁-C₄)(alkyl)acrylate; and
   iii) one or more hydrocarbon-based oil(s), followed by
2) the application to said materials of a composition (B), which is preferably anhydrous, comprising:
   iv) one or more amine compound(s) chosen from:
      e) polyamine compounds bearing several primary amine and/or secondary amine groups, and
      f) amino alkoxysilanes,

it being understood that the process of the invention uses v) one or more cosmetic active agents chosen from a) pigments, b) active agents for caring for keratin materials, notably the skin, and c) UV-screening agents, and also d) mixtures thereof, ingredients a) to d) possibly being found in composition (A), and/or in composition (B), and/or in another composition (C).

This process for treating keratin materials in at least two successive steps using, during a first step, the oily dispersion (A) as defined above and then, during a following step, a composition (B) comprising one or more amine compounds as defined above and optionally a composition (C), makes it possible to obtain a treatment of said keratin materials that is notably resistant to sweat, to shower gels, to water and to fatty substances, in particular plant or animal food oils, and more particularly plant oils such as olive oil, sunflower oil, walnut oil, hazelnut oil, etc.

The keratin material treatment process of the present patent application is also suitable for making up the skin or the lips, such as foundations and lipsticks.

A subject of the present patent application is also a process, notably a cosmetic process, for caring for or making up the skin or the lips, more particularly facial skin, in particular wrinkled skin, comprising the topical application to the skin or the lips of the compositions, notably the cosmetic compositions, (A) and (B) and optionally (C) as described previously. A subject of the invention is also a process, notably a cosmetic process, for caring for the skin, more particularly facial skin, in particular wrinkled skin, comprising the topical application to the skin of the compositions, notably the cosmetic compositions, (A) and (B) and optionally (C), as described previously.

The process of the present patent application is in particular intended for smoothing human facial and/or bodily skin and/or for reducing or eliminating the signs of skin aging, in particular for reducing or eliminating wrinkles and/or fine lines of the skin.

A subject of the invention is also the cosmetic use, as a skin tensioning agent, in particular for wrinkled skin, of the compositions, notably the cosmetic compositions, (A) and (B) and optionally (C) as described previously.

The term “tensioning agent means compounds that are capable of having a noticeable tensioning effect, i.e. of smoothing out the skin and immediately, reducing the wrinkles and fine lines, or even making them disappear.

The tensioning effect may be characterized by an in vitro shrinkage test.

For the purposes of the present invention and unless otherwise indicated:
an “alkyl radical” is a linear or branched saturated C₁-C₈, in particular C₁-C₆, preferably C₁-C₄ hydrocarbon-based group such as methyl, ethyl, isopropyl and tert-butyl;
an “alkylene radical” is a linear or branched divalent saturated C₁-C₈, in particular C₁-C₆, preferably C₁-C₄ hydrocarbon-based group such as methylene, ethylene or propylene;
a “cycloalkyl” radical is a cyclic saturated hydrocarbon-based group comprising from 1 to 3 rings, preferably 2 rings, and comprising from 3 to 13 carbon atoms, preferably between 5 and 10 carbon atoms, such as cyclopentyl, cyclohexyl, cycloheptyl, norbornyl, or isobornyl, the cycloalkyl radical possibly being substituted with one or more (C₁-C₄)alkyl groups such as methyl; preferably, the cycloalkyl radical is an isobornyl group.
a “cyclic” radical is a cyclic saturated or unsaturated, aromatic or non-aromatic hydrocarbon-based group comprising from 1 to 3 rings, preferably 1 ring, and comprising from 3 to 10 carbon atoms, such as cyclohexyl or phenyl;
an “aryl” radical is a cyclic unsaturated aromatic hydrocarbon-based radical comprising from 6 to 12 carbon atoms, which is mono- or bicyclic, fused or unfused; preferably, the aryl group comprises 1 ring and to 6 carbon atoms, such as phenyl;
an “aryloxy” radical is an aryl-oxy, i.e. aryl-O—, radical, with aryl as defined previously, preferably phenoxy;
an “aryl(C₁-C₄)alkoxy” radical is an aryl-(C₁-C₄)alkyl-O— radical, preferably benzoxy.

The Oily Dispersion (A)

The process of the invention involves at least one oily dispersion (A) of i) particles of at least one polymer surface-stabilized with ii) at least one stabilizer in a preferably anhydrous medium, also containing iii) at least one hydrocarbon-based oil.

Moreover, the dispersions according to the invention are constituted of particles, which are generally spherical, of at least one surface-stabilized polymer, in a non-aqueous medium.

i) Polymer Particles

The particle(s) of the dispersion of the process of the invention are constituted of one or more ethylenic copolymers of a) (C₁-C₄)alkyl (C₁-C₄)(alkyl)acrylate and of b) ethylenically unsaturated anhydride compound.

The term “ethylenic copolymer” means a polymer resulting from the polymerization of two monomers: of the monomer a) (C₁-C₄)alkyl (C₁-C₄)(alkyl)acrylate and of the monomer b) of ethylenically unsaturated anhydride compound.

The term “ethylenically unsaturated anhydride compound” means a carboxylic acid anhydride compound comprising at least one ethylenic unsaturation —(Rₐ)C═C(R_{b})—, —C(Rₐ)═C(R_{b})—R_{c} or >C=C(Rₐ)—R_{b}, with Rₐ, R_{b}, and R_{c}, which may be identical or different, representing a hydrogen atom or a (C₁-C₄)alkyl group such as methyl, preferably hydrogen. In particular, the ethylenically unsaturated anhydride compound is a cyclic compound, which is preferably 5- or 6-membered, and comprising an ethylenic unsaturation.

According to a preferred embodiment of the invention, the polymer constituting the particles i) is a copolymer of acrylate:

a) of formula H₂C═C(R)—C(O)—O—R′ with R representing a hydrogen atom or a (C₁-C₄)alkyl group such as methyl, and R′ representing a linear or branched, preferably linear, (C₁-C₄)alkyl and in particular (C₁-C₃)alkyl group such as methyl or ethyl, preferably linear or branched, preferably linear, C₁-C₄ alkyl (meth)acrylate, in particular C₁-C₃ alkyl (meth)acrylate; and

b) of an ethylenically unsaturated anhydride monomer.

Particularly, the polymer of the particles is a polymer of linear or branched, preferably linear, C₁-C₄ alkyl (meth)acrylate, in particular linear C₁-C₃ alkyl (meth)acrylate, and of ethylenically unsaturated anhydride monomer.

The monomers a) are preferably chosen from methyl (meth)acrylate, ethyl (meth)acrylate, n-propyl (meth)acrylate, isopropyl (meth)acrylate, n-butyl (meth)acrylate and tert-butyl (meth)acrylate.

Advantageously, a linear or branched, preferably linear, C₁-C₄ alkyl acrylate, in particular (C₁-C₃)alkyl acrylate, monomer is used. Preferentially, a) is chosen from methyl acrylate and ethyl acrylate.

The polymer of the particles also comprises an ethylenically unsaturated anhydride monomer b).

Preferentially, the ethylenically unsaturated anhydride compound(s) b) of the invention are chosen from derivatives of maleic anhydride (Ib) and of itaconic anhydride (IIb):

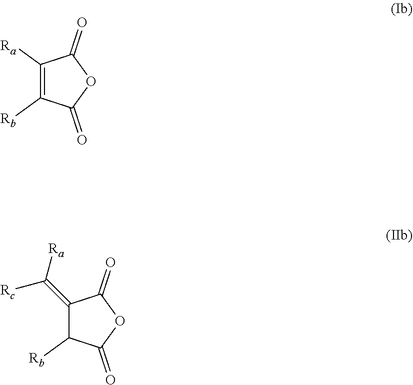

in which formulae (Ib) and (IIb) Rₐ, R_{b} and R_{c}, which may be identical or different, represent a hydrogen atom or a (C₁-C₄)alkyl group; preferably, Rₐ, R_{b}, and R_{c} represent a hydrogen atom.

More preferentially, the ethylenically unsaturated anhydride monomer of the invention is of formula (Ib) and even more preferentially is maleic anhydride.

According to a preferred embodiment of the invention, the polymer(s) of the particles i) comprise, or essentially consist of, from 80% to 99.99% by weight of monomer a) and from 0.01% to 20% by weight of monomer b), relative to the total weight of the polymer.

According to a preferred embodiment of the invention, the copolymer(s) of the particles i) of dispersion (A) comprise from 80% to 99.99% by weight of ingredient a) and from 0.01% to 20% by weight of ingredient b), relative to the total weight of the copolymer(s) or particles. The preferred amount of a) is between 80% and 99.99% by weight relative to the total weight of the copolymer(s) (or of the particle(s)), in particular between 85% and 98%, more preferentially between 87% and 94% by weight relative to the total weight of the copolymer(s).

The polymer of the particles may be chosen from:
methyl acrylate/maleic anhydride copolymers;
ethyl acrylate/maleic anhydride copolymers; and
methyl acrylate/ethyl acrylate/maleic anhydride copolymers.

Advantageously, the polymer of the particles is a non-crosslinked polymer.

The polymer of the particles of the dispersion preferably has a number-average molecular weight ranging from 2000 to 10 000 000.

The polymer of the particles may be present in the dispersion in an amount ranging from 20% to 60% by weight relative to the total weight of the dispersion (A), in particular between 21% and 58.5% by weight relative to the total weight of the dispersion (A), preferably ranging from 30% to 50% by weight relative to the total weight of the dispersion (A), more preferentially ranging from 36% to 42% by weight relative to the total weight of the dispersion (A).

Preferably, the particle(s) consist of copolymer a) and b) which constitute the core of said particle.

According to a particular embodiment of the invention, the particles i) consist of copolymers a) and b) with an a)/b) weight ratio inclusively between 5.5 and 20, preferably between 6.5 and 16 and even more preferentially between 6.6 and 15.6.

ii) The Stabilizer(s)

The dispersion (A) according to the invention also comprises one or more stabilizers ii). Preferably, only one type of stabilizer ii) is used in the invention.

The stabilizer(s) of the invention are constituted of ethylenic polymers chosen from c) polymers of (C₃-C₁₂)cycloalkyl (C₁-C₆)(alkyl)acrylate monomers; and d) copolymers of (C₃-C₁₂)cycloalkyl (C₁-C₆)(alkyl)acrylate and (C₁-C₄)alkyl (C₁-C₄)(alkyl)acrylate.

According to a preferred embodiment of the invention, the stabilizer(s) ii) are constituted of ethylenic polymers chosen from

c) polymers of monomers of formula H₂C═C(R)—C(O)—O—R″ with R representing a hydrogen atom or (C₁-C₄)alkyl group such as methyl, and R″ representing a (C₅-C₁₀)cycloalkyl group such as norbornyl or isobornyl, preferably isobornyl; and

d) copolymers of H₂C=C(R)—C(O)—O—R′ and of H₂C═C(R)—C(O)—O—R″ with R, R′ and R″ as defined previously.

Particularly, the stabilizer ii) is an isobornyl (meth)acrylate polymer chosen from isobornyl (meth)acrylate homopolymer and statistical copolymers of isobornyl (meth)acrylate and C₁-C₄ alkyl (meth)acrylate present in an isobornyl (meth)acrylate/C₁-C₄ alkyl (meth)acrylate weight ratio of greater than 4; advantageously, said weight ratio ranges from 4.5 to 19.

For these statistical copolymers, the defined weight ratio makes it possible to obtain a polymer dispersion that is stable, notably after storage for seven days at room temperature.

Advantageously, the stabilizer is chosen from:
isobornyl acrylate homopolymers,
statistical copolymers of isobornyl acrylate/methyl acrylate,
statistical copolymers of isobornyl acrylate/ethyl acrylate; and
statistical copolymers of isobornyl acrylate/methyl acrylate/ethyl acrylate according to the weight ratio described previously.

Advantageously, the sum ii) of stabilizer(s)+i) polymer particle(s) present in the dispersion (A) comprises from 10% to 50% by weight of copolymers d) and from 50% to 90% by weight of polymers c), relative to the total weight of the sum ii) of stabilizer(s)+i) polymer particle(s).

Preferentially, the sum ii) of stabilizer(s)+ii) polymer particle(s) present in the dispersion comprises from 15% to 30% by weight of copolymers d) and from 70% to 85% by weight of polymers c), relative to the total weight of the sum ii) of stabilizer(s)+i) polymer particle(s).

ii) The Hydrocarbon-Based Oil(s)

The dispersion (A) according to the invention comprises one or more identical or different, preferably identical, hydrocarbon-based oils.

The term “oil” means a fatty substance that is liquid at room temperature (25° C.) and at atmospheric pressure.

The term “hydrocarbon-based oil” means an oil formed essentially from, or even constituted of, carbon and hydrogen atoms, and optionally of oxygen and nitrogen atoms, and not containing any silicon or fluorine atoms. It may contain hydroxyl, ester, ether, carboxylic acid, amine and/or amide groups.

The hydrocarbon-based oil may be volatile or nonvolatile.

According to a preferred embodiment of the invention, the hydrocarbon-based oil(s) are volatile or are a mixture of different volatile oils, more preferentially chosen from isododecane and octyldodecanol.

According to a particular embodiment, the hydrocarbon-based oil(s) are a mixture of a volatile oil and a nonvolatile oil.

The term “volatile oil” means an oil (or non-aqueous medium) that can evaporate on contact with the skin in less than one hour, at room temperature and at atmospheric pressure. The volatile oil is a volatile cosmetic oil, which is liquid at room temperature, notably having a nonzero vapor pressure, at room temperature and at atmospheric pressure, in particular having a vapor pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

The term “nonvolatile oil” means an oil with a vapor pressure of less than 0.13 Pa.

Volatile silicone oils that may be mentioned include volatile linear or cyclic silicone oils, notably those with a viscosity s 8 centistokes (cSt) (8×10⁻⁶ m²/s), and notably containing from 2 to 10 silicon atoms and in particular from 2 to 7 silicon atoms, these silicones optionally including alkyl or alkoxy groups containing from 1 to 10 carbon atoms. As volatile silicone oils that may be used in the invention, mention may notably be made of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctytrisiloxane, hexamethyldisiloxane, octamethyltrisiloxane, decamethyltetrasiloxane and dodecamethylpentasiloxane, and mixtures thereof.

As nonvolatile silicone oils, mention may be made of linear or cyclic nonvolatile polydimethylsiloxanes (PDMSs); polydimethylsiloxanes including alkyl, alkoxy and/or phenyl groups, which are pendent or at the end of a silicone chain, these groups containing from 2 to 24 carbon atoms; phenyl silicones, for instance phenyl timethicones, phenyl dimethicones, phenyltrimethylsiloxydiphenylsiloxanes, diphenyl dimethicones, diphenylmethyldiphenyltisiloxanes, 2-phenylethyl trimethylsiloxysilicates and pentaphenyl silicone oils. The hydrocarbon-based oil may be chosen from:

hydrocarbon-based oils containing from 8 to 14 carbon atoms, and notably:
branched C₈-C₁₄ alkanes, such as C₈-C₁₄ isoalkanes of petroleum origin (also known as isoparaffins), such as isododecane (also known as 2,2,4,4,6-pentamethylheptane), isodecane and, for example, the oils sold under the Isopar or Permethyl trade names,
linear alkanes, for instance n-dodecane (C12) and n-tetradecane (C14) sold by Sasol under the respective references Parafol 12-97 and Parafol 14-97, and also mixtures thereof, the undecane-tridecane mixture, the mixtures of n-undecane (C11) and of n-tridecane (C13) obtained in examples 1 and 2 of patent application WO 2008/155 059 from the company Cognis, and mixtures thereof,
short-chain esters (containing from 3 to 8 carbon atoms in total) such as ethyl acetate, methyl acetate, propyl acetate or n-butyl acetate.
hydrocarbon-based oils of plant origin such as triglycerides constituted of fatty acid esters of glycerol, the fatty acids of which may have chain lengths ranging from C₄ to C₄, these chains possibly being linear or branched, and saturated or unsaturated; these oils are notably heptanoic acid or octanoic acid triglycerides, or alternatively wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cotton oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil or musk rose oil; shea butter; or else caprylic/capric acid triglycerides, for instance those sold by the company Stéarinerie Dubois or those sold under the names Miglyol 810e, 812e and 818e by the company Dynamit Nobel,
synthetic ethers containing from 10 to 40 carbon atoms,
linear or branched hydrocarbons of mineral or synthetic origin, such as petroleum jelly, polydecenes, hydrogenated polyisobutene such as Parleam®, squalane and liquid paraffins, and mixtures thereof,
synthetic esters such as oils of formula R₁C(O)—O—R₂ in which R₁ represents a linear or branched fatty acid residue including from 1 to 40 carbon atoms and R₂ represents a, notably branched, hydrocarbon-based chain containing from 1 to 40 carbon atoms, on condition that R₁+R₂≥10, for instance purcellin oil (cetostearyl octanoate), isopropyl myristate, isopropyl palmitate, C₁₂ to C₁ alkyl benzoates, hexyl laurate, diisopropyl adipate, isononyl isononanoate, 2-ethylhexyl palmitate, isostearyl isostearate, 2-hexyldecyl laurate, 2-octyldecyl palmitate, 2-octyldodecyl myristate, alcohol or polyalcohol heptanoates, octanoates, decanoates or ricinoleates such as propylene glycol dioctanoate; hydroxylated esters such as isostearyl lactate, diisostearyl malate and 2-octyldodecyl lactate; polyol esters and pentaerythritol esters,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol and 2-undecylpentadecanol.

In addition to the hydrocarbon-based oil, the dispersion (A) may comprise a silicone oil. If silicone oil is in the dispersion (A), it is preferably in an amount which does not exceed 10% by weight relative to the weight of the dispersion (A), more particularly in an amount of less than 5% and preferentially 2%. The term “silicone oil” means an oil comprising at least one silicon atom and notably at least one Si—O group. The silicone oil may be volatile or nonvolatile.

According to a particular embodiment, the dispersion (A) comprises a hydrocarbon-based oil in a content ranging from 60% to 100% by weight relative to the total weight of the oils present in the composition and from 0 to 40% by weight of silicone oil. According to a preferred embodiment of the invention, the composition contains, as oil, only a hydrocarbon-based oil.

Advantageously, the hydrocarbon-based oil(s) of the invention are apolar, i.e. formed solely of carbon and hydrogen atoms.

The hydrocarbon-based oil(s) are preferably chosen from hydrocarbon-based oils containing from 8 to 14 carbon atoms, which are in particular volatile, more particularly the apolar oils, described previously.

Preferentially, the hydrocarbon-based oil(s) of the invention are isododecane.

According to another advantageous embodiment of the invention, the hydrocarbon-based oil(s) are a mixture of nonvolatile and volatile oil; preferably, the mixture comprises isododecane as volatile oil. In particular, in the mixture, the nonvolatile oil is a phenyl silicone oil, preferably chosen from pentaphenyl silicone oils.

According to a particular embodiment of the invention, the compositions used in the process, (B) and (C), also comprise at least one oil as defined previously, notably a hydrocarbon-based oil.

The polymer particles of the dispersion preferably have a number-mean size ranging from 5 to 500 nm, notably ranging from 10 to 400 nm and better still ranging from 20 to 300 nm.

Method for Preparing the Dispersion (A)

Without this being limiting, in general, the dispersion according to the invention may be prepared in the following manner: The polymerization is performed in “dispersion”, i.e. by precipitation of the polymer being formed, with protection of the particles formed with one or more stabilizers, preferably one stabilizer.
In a first step, the stabilizing polymer (or stabilizer ii)) is prepared by mixing the constituent monomer(s) of the stabilizing polymer c) or d) with iv) a free-radical initiator, in a solvent known as the synthesis solvent, and by polymerizing these monomers;
In a second step, the constituent monomer(s) of the polymer of the particles i) are added to the stabilizing polymer formed in the preceding step and polymerization of these added monomers is performed in the presence of the free-radical initiator.

When the nonaqueous medium is a nonvolatile hydrocarbon-based oil iii), the polymerization may be performed in an apolar organic solvent (synthesis solvent), followed by adding the nonvolatile hydrocarbon-based oil (which should be miscible with said synthesis solvent) and selectively distilling off the synthesis solvent.

The cosmetic agent(s), preferably the pigment(s), are added during the first step. According to another variant, the cosmetic agent(s), preferably the pigment(s), are added during the second step or after the second step.

A synthesis solvent which is such that the monomers of the stabilizing polymer and the free-radical initiator are soluble therein, and the polymer particles obtained are insoluble therein, so that they precipitate therein during their formation, is thus chosen.

In particular, the synthesis solvent chosen is one which is apolar and organic, preferably chosen from alkanes such as heptane or cyclohexane.

When the nonaqueous medium is a volatile hydrocarbon-based oil iii), the polymerization may be performed directly in said oil, which thus also acts as synthesis solvent. The monomers should also be soluble therein, as should the free-radical initiator, and the polymer of the particles which is obtained should be insoluble therein.

The cosmetic agent(s) v), preferably the pigment(s), may be added during the first step. According to another variant, the dye(s) and/or pigment(s) are added during the second step or after the second step.

A synthesis solvent which is such that the monomers of the stabilizing polymer and the free-radical initiator are soluble therein, and the polymer particles obtained are insoluble therein, so that they precipitate therein during their formation, is thus chosen.

In particular, the synthesis solvent chosen is one which is apolar and organic, preferably chosen from alkanes such as heptane or cyclohexane.

When the nonaqueous medium is a volatile hydrocarbon-based oil iii), the polymerization may be performed directly in said oil, which thus also acts as synthesis solvent. The monomers should also be soluble therein, as should the free-radical initiator, and the polymer of the particles which is obtained should be insoluble therein.

The monomers are preferably present in the synthesis solvent, before polymerization, in a proportion of 5% to 45% by weight. The total amount of the monomers may be present in the solvent before the start of the reaction, or a portion of the monomers may be added gradually as the polymerization reaction proceeds.

The polymerization is preferentially performed in the presence vi) of one or more free-radical initiators, notably of the type such as:
peroxide, in particular chosen from tert-butyl peroxy-2-ethylhexanoate: Trigonox 21S; 2,5-dimethyl-2,5-bis(2-ethylhexanoylperoxy)hexane:Trigonox 141; tert-butyl peroxypivalate: Trigonox 25C75 from AkzoNobel; or
azo, in particular chosen from AIBN: azobisisobutyronitrile; V50: 2,2′-azobis(2-amidinopropane) dihydrochloride.

The polymerization is preferably performed at a temperature ranging from 70 to 110° C. and at atmospheric pressure.

The polymer particles i) are surface-stabilized, when they are formed during the polymerization, by means of the stabilizer ii).

The stabilization may be performed by any known means, and in particular by direct addition of the stabilizer ii), during the polymerization.

The stabilizer ii) is preferably also present in the mixture before polymerization of the monomers of the polymer of the particles i). However, it is also possible to add it continuously, notably when the monomers of the polymer of the particles i) are also added continuously.

From 10% to 30% by weight and preferably from 15% to 25% by weight of the stabilizer(s) may be used relative to the total weight of monomers used (stabilizers ii)+polymer particles i)).

The polymer particle dispersion (A) advantageously comprises from 30% to 65% by weight of solids relative to the total weight of said dispersion and preferably from 40% to 60% by weight relative to the total weight of said dispersion.

The composition according to the invention preferably comprises a content of polymers of particle i)+dispersing polymers ii) ranging from 1% to 50% by weight and preferably ranging from 2% to 30% by weight relative to the total weight of composition (A).

According to a preferred embodiment of the invention, the dispersion (A) according to the invention is an anhydrous composition.

The term “anhydrous” dispersion or composition refers to a dispersion or composition containing less than 2% by weight of water, or even less than 0.5% of water, and notably free of water. Where appropriate, such small amounts of water may notably be introduced by ingredients of the composition that may contain residual amounts thereof.

According to another embodiment of the present patent application, the dispersion (A) is in inverse emulsion, i.e. of water-in-oil type (W/O). In this case, the composition comprises one or more surfactants, which are preferably nonionic. The inverse emulsions of (A) are preferably chosen in makeup, notably in mascaras.

The Composition (B)

Composition B of the process of the invention comprises one or more amine compounds iv).

iv) The Amine Compound(s):

The amine compounds used in the process of the invention are chosen from:

e) polyamines bearing several primary amine and/or secondary amine groups, and

f) amino alkoxysilanes.

The amine compound(s) used in the process according to the invention are notably chosen from amino alkoxysilane compounds, diamine compounds and triamine compounds.

According to a particular embodiment of the invention, the polyamine compound(s) particularly comprise from 2 to 20 carbon atoms; the polyamine compound(s) are notably nonpolymeric.

The term ‘nonpolymeric’ compound refers to one or more compounds which are not directly obtained via a monomer polymerization reaction.

Polyamine compounds that may notably be mentioned include N-methyl-1,3-diaminopropene, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine, lysine, cystamine, xylenediamine, tris(2-aminoethyl)amine and spermidine.

According to another particular embodiment of the invention, the amine compound(s) iv) are chosen from f) amino alkoxysilanes such as those of formula (IVa):

R′₁—Si(OR′₂)_{z}(R′₃)ₓ (IVa)

in which formula (IVa):
R′₁ is a linear or branched, saturated or unsaturated, cyclic or acyclic C₁-C₁₀ hydrocarbon-based chain substituted with one or more groups chosen from the following groups:
primary amine NH₂ or secondary amine N(H)R with R representing a (C₁-C₄)alkyl group,
aryl or aryloxy substituted with an amino or (C₁-C₄)alkylamino group or with a C₁-C₄ aminoalkyl group, and
aldehyde —C(O)—H, carboxyl —C(O)—OH, amide —C(O)—NH₂ or urea —NH—C(O)—NH₂; R′₁ is optionally interrupted in its hydrocarbon-based chain with one or more heteroatoms (notably O, S. NH), a carbonyl group (CO), or a combination thereof, such as ester-C(O)—O—, or amide —C(O)—NH—, R′₁ being bonded directly to the silicon atom via a carbon atom,
R′₂ and R′₃ which may be identical or different, represent a linear or branched alkyl group comprising from 1 to 6 carbon atoms,
z denotes an integer ranging from 1 to 3,
and x denotes an integer ranging from 0 to 2, with z+x=3.

Preferably, R′₂ represents an alkyl group comprising from 1 to 4 carbon atoms.

Preferably, R′₂ represents a linear alkyl group comprising from 1 to 4 carbon atoms.

Preferably, R′₂ represents an ethyl group.

Preferably, R′₃ represents an alkyl group comprising from 1 to 4 carbon atoms.

Preferably, R′₃ represents a linear alkyl group comprising from 1 to 4 carbon atoms.

Preferably. R′₃ represents a methyl or ethyl group.

Preferably, R′₁ is an acyclic chain.

Preferably, R′₁ is a linear or branched, saturated or unsaturated C₁-C₆ hydrocarbon-based chain substituted with an amine NH₂ or N(H)R group, with R representing a C₁-C₆ alkyl group, C₃-C₆ cycloalkyl, or C₆ aromatic group.

Preferentially, R′₁ is a saturated linear C₂-C₄ hydrocarbon-based chain substituted with an amine group NH₂.

More preferentially, R′₁ is a saturated linear C₂-C₄ hydrocarbon-based chain substituted with an amine group NH₂.

Preferably, R′₁ is a saturated linear C₁-C₆ hydrocarbon-based chain substituted with an amine group NH₂.

R′₂ represents an alkyl group comprising from 1 to 4 carbon atoms,

R′₃ represents an alkyl group comprising from 1 to 4 carbon atoms.

Preferably, z is equal to 3.

Preferably, the amino alkoxysilane of formula (IVa) is chosen from 3-aminopropyltriethoxysilane (APTES), 3-aminoethyltriethoxysilane (AETES), 3-aminopropylmethyldiethoxysilane, N-(2-aminoethyl)-3-aminopropyttriethoxysilane, 3-(m-aminophenoxy)propyltrimethoxysilane, p-aminophenyltrimethoxysilane and N-(2-aminoethylaminomethyl)phenethyltrimethoxysilane.

Preferably, the amino alkoxysilane (IVa) is chosen from 3-aminopropyttriethoxysilane (APTES), 3-aminoethyltriethoxysilane (AETES), 3-aminopropylmethyldiethoxysilane and N-(2-aminoethyl)-3-aminopropyltriethoxysilane.

Preferably, the amino alkoxysilane (IVa) is 3-aminopropyltriethoxysilane (APTES).

Preferably, the amine compound(s) are chosen from 3-aminopropyltriethoxysilane (APTES), N-methyl-1,3-diaminopropane, N-propyl-1,3-diaminopropane, N-isopropyl-1,3-diaminopropane, N-cyclohexyl-1,3-diaminopropane, 2-(3-aminopropylamino)ethanol, 3-(2-aminoethyl)aminopropylamine, bis(3-aminopropyl)amine, methylbis(3-aminopropyl)amine, N-(3-aminopropyl)-1,4-diaminobutane, N,N-dimethyldipropylenetriamine, 1,2-bis(3-aminopropylamino)ethane, N,N′-bis(3-aminopropyl)-1,3-propanediamine, ethylenediamine, 1,3-propylenediamine, 1,4-butylenediamine and lysine.

Preferentially, the amine compound is chosen from ethylenediamine, 1,3-propylenediamine, 1,4-butylenediamine and 3-aminopropyltriethoxysilane (APTES).

More preferentially, the amine compound is ethylenediamine or 3-aminopropyltriethoxysilane (APTES).

The amine compound(s) may also be chosen from e) polyamines bearing several primary amine and/or secondary amine groups and in particular amine polymers, notably having a weight-average molecular weight ranging from 500 to 1 000 000, preferably ranging from 500 to 500 000, and preferentially ranging from 500 to 100 000.

As amine or polyamine polymers e), use may be made of poly((C₂-C₅)alkyleneimines), and in particular:
polyethyleneimines and polypropyleneimines, notably poly(ethyleneimine)s (for example the product sold under the reference 46.852-3 by the company Aldrich Chemical);
poly(allylamine) (for example the product sold under the reference 47,913-6 by the company Aldrich Chemical);
polyvinylamines and copolymers thereof, in particular with vinylamides; mention may notably be made of vinylamine/vinylformamide copolymers, such as those sold under the name Lupamin® 9030 by the company BASF;
polyamino acids containing NH₂ groups such as polylysine, for example the product sold by the company JNC Corporation (formerly Chisso); aminodextran, such as the product sold by the company CarboMer Inc;
amino polyvinyl alcohol, such as the product sold by the company CarboMer Inc, copolymers based on acrylamidopropylamine;
chitosans; and
polydi(C₁-C₄)alkylsiloxanes, in particular polydimethylsiloxanes, comprising amine groups at the chain end or on side chains are particularly end or side amino(C₁-C₆)alkyl groups such as aminopropyl, more particularly those of formula (IVb) or (IVc) or (IVd):

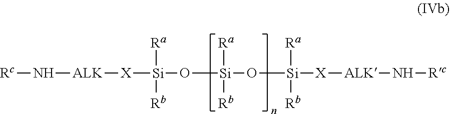

in which formula (IVb) R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl, R^{c} and R′^{c}, which may be identical or different, preferably identical, represent a hydrogen atom, a (C₁-C₄)alkyl group, an amino(C₁-C₄)alkyl group or a (C₁-C₄)alkylamino(C₁-C₄)alkyl group, preferably a hydrogen atom or an amino(C₁-C₄)alkyl group such as aminoethyl; X represents a covalent bond, an oxygen atom, preferably a covalent bond, ALK and ALK′, which may be identical or different, preferably identical, represent a (C₁-C₆)alkylene group, preferably (C₁-C₄)alkylene group such as propylene; n representing an integer greater than 2 and more particularly the value of n is such that the weight-average molecular weight of the silicone is between 500 and 55 000;
Preferentially, the polydi(C₁-C₄)alkylsiloxanes of formula (IVb) are of formula (IV′b) or (IV″b) below:

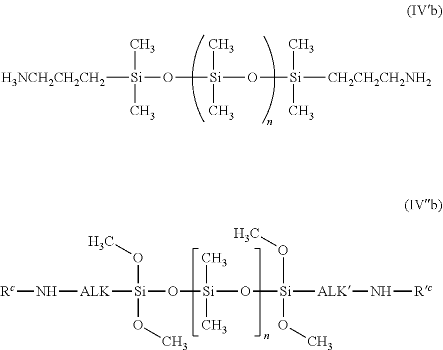

in which formula (IVb) the value of n is such that the weight-average molecular weight of the silicone is between 500 and 55 000. As examples of aminosilicones (IVb) or (IV′b), mention may be made of those sold under the names DMS-A11, DMS-A12, DMS-A15. DMS-A21, DMS-A31, DMS-A32 and DMS-A35 by the company Gelest;
formula (IV″b) with R^{c}, R′^{c}, ALK, ALK′, and n as defined previously for (IVb). Preferably, ALK and ALK′ are identical and represent a (C₁-C₄)alkylene group such as propylene, R^{c} and R′^{c} are identical and represent an amino(C₁-C₄)alkyl group such as aminoethyl
mention may be made particularly of Dimethoxysilyl Ethylenediaminopropyl Dimethicone (RN: 71750-80-6), under the trade name GP-RA-157, sold by Genesee Polymers.

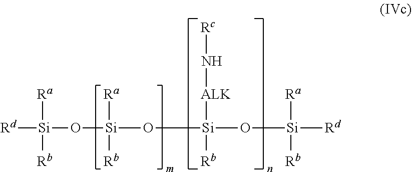

in which formula (IVc) R^{a}, R^{b}, and R^{d}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl, R^{d} may also represent a (C₁-C₆)alkyl group substituted with a (C₁-C₄)alkylamino or amino group, R^{c} represents a hydrogen atom or a (C₁-C₄)alkyl group, preferably a hydrogen atom; ALK represents a (C₁-C₆)alkylene group, preferably (C₁-C₄)alkylene such as propylene; n and m, which may be identical or different, represent an integer greater than 2 and more particularly the values of m and n are such that the weight-average molecular weight of the silicone is between 1000 and 55 000;
Preferentially, the polydi(C₁-C₄)alkylsiloxanes of formula (IVc) have the formula (IV′c) below:

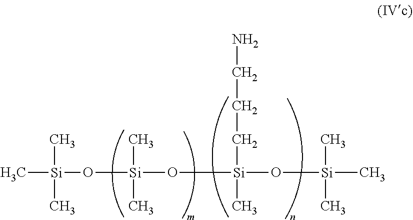

in which formula (V′c) the values of n and m are such that the weight-average molecular weight of the silicone is between 1000 and 55 000. As examples of silicones (IVc), mention may be made of those sold under the names AMS-132, AMS-152, AMS-162, AMS-163, AMS-191 and AMS-1203 by the company Gelest;

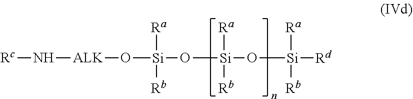

in which formula (IVd) R^{a} and R^{b}, which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl, and R^{d} represents a (C₁-C₆)alkyl group optionally substituted with a (C₁-C₄)alkylamino or amino group, preferably (C₁-C₄)alkyl such as isobutyl, tert-butyl or n-butyl, R^{c} represents a hydrogen atom or a (C₁-C₄)alkyl group, preferably a hydrogen atom; ALK represents a (C₁-C₆)alkylene group, preferably (C₁-C₄)alkylene such as propylene, n representing an integer greater than 2 and more particularly the value of n is such that the weight-average molecular weight of the silicone is between 500 and 5000;
Preferentially, the polydi(C₁-C₄)alkylsiloxanes of formula (IVd) have the formula (IV′d) below:

H₂NCH₂CH₂CH₂—Si(CH₃)₂—O—[Si(CH₃)₂—O]n-Si(CH₃)₂C₄H₉ (IV′d)
in formula (IV′d), the value of n is such that the weight-average molecular weight of the silicone is between 500 and 3000. As examples of silicones (IVd), mention may be made of the products sold under the names MCR-A11 and MCR-A12 by the company Gelest;
amodimethicones of formula (IVe):

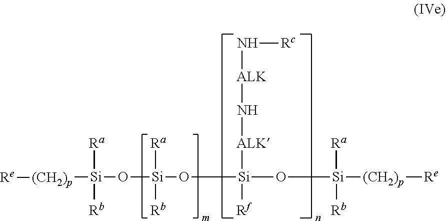

in which formula (IVe)
R^{a} and R^{b} which may be identical or different, preferably identical, represent a group from among: (C₁-C₄)alkyl such as methyl, (C₁-C₄)alkoxy such as methoxy, aryl such as phenyl, aryloxy such as phenoxy, aryl(C₁-C₄)alkyl such as benzyl, or aryl(C₁-C₄)alkoxy such as benzoxy, preferably (C₁-C₄)alkyl such as methyl,
R^{c} represents a hydrogen atom or a (C₁-C₄)alkyl group, preferably a hydrogen atom;
R^{e} represent a hydroxyl, (C₁-C₄)alkoxy, amino or (C₁-C₄)alkylamino group,
R^{f} represents a (C₁-C₄)alkyl group such as methyl, a (C₁-C₄)alkoxy group such as methoxy, a hydroxyl or —O—(SiR₂)ₓR′ group with R representing a (C₁-C₄)alkyl or (C₁-C₄)alkoxy group and R′ representing a (C₁-C₄)alkoxy or hydroxyl group; preferably,
R^{f} represents a (C₁-C₄)alkyl, (C₁-C₄)alkoxy or —O—(SiR₂)ₓR′ group with R representing a (C₁-C₄)alkyl group such as methyl and R′ a hydroxyl or (C₁-C₄)alkoxy group such as methoxy;
R^{g} represents a hydrogen atom or a (C₁-C₆)alkyl group,
ALK and ALK′, which may be identical or different, represent a (C₁-C₆)alkylene group, preferably (C₁-C₄)alkylene such as ethylene or propylene; n and m, which may be identical or different, represent an integer greater than 2, p and x are integers greater than or equal to 0; preferably, p is between 2 and 20 and more particularly the values of m, n, p and x are such that the weight-average molecular weight of the silicone is between 2000 and 700 000, preferentially between 5000 and 500 000;
Preferentially, the amodimethicones of formula (IVe) are of formula (IV′e) below:

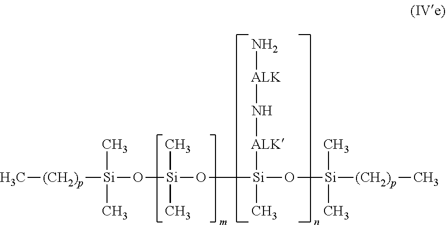

in which formula (IV′e) ALK represents a (C₁-C₆)alkylene group, preferably ethylene, ALK′ represents a (C₁-C₆)alkylene group, preferably propylene, and m, n and p represent greater than 2, with m, n and p such that the weight-average molecular mass of the compound is between approximately 5000 and 500 000; preferably, p represents an integer between 8 and 20;
according to another preferred embodiment, the amodimethicones of formula (IVe) are chosen from the compound of formula

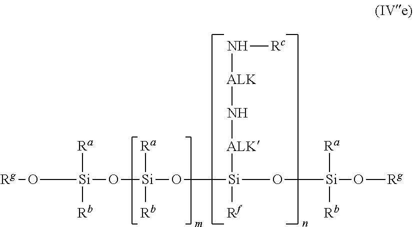

in which formula (IV″e)
R^{a} and R^{b}, which may be identical or different, preferably identical, represent a (C₁-C₄)alkyl group such as methyl, a (C₁-C₄)alkoxy group such as methoxy, preferably a (C₁-C₄)alkyl group such as methyl,
R^{c} represents a hydrogen atom or a (C₁-C₄)alkyl group, preferably a hydrogen atom;
R^{g} represents a hydrogen atom or a (C₁-C₄)alkyl group;
R^{f} represents a (C₁-C₄)alkyl group such as methyl, a (C₁-C₄)alkoxy group such as methoxy, or —O—(SiR₂)ₓ—R′ with R representing a (C₁-C₄)alkyl group such as methyl and R′ a hydroxyl or (C₁-C₄)alkoxy group such as methoxy;
R^{g} represents a hydrogen atom or a (C₁-C₆)alkyl group,
ALK represents a (C₁-C₆)alkylene group, preferably ethylene,
ALK′ represents a (C₁-C₆)alkylene group, preferably propylene,
n and m, which may be identical or different, representing an integer greater than 2,
x is an integer greater than or equal to 0; preferably, the values of m, n and x are such that the weight-average molecular weight of the silicone is between 2000 and 700 000, preferentially between 5000 and 500 000;
even more particularly, (IV″e) represents:

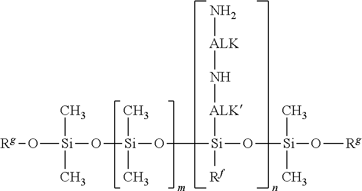

with R^{f}, R^{g}, ALK, ALK′, m, n as defined for (IV″e). The amodimethicones and trimethylsiloxyamodimethicones belonging to formula (IV″e) and the formula above are, for example, the amodimethicones and trimethylsiloxyamodimethicones of ADM type sold by the company Wacker-Belsil®; mention may also be made of polydimethylsiloxanes bearing aminoethylaminopropyl groups, bearing a methoxy and/or hydroxyl function and α-ω silanols as a cationic 60% aqueous emulsion (supplier reference: Xiameter MEM-8299 Emulsion by Dow Corning or under the supplier reference: Belsil ADM 4000 E by Wacker);
Polydimethylsiloxane bearing aminoethyl iminopropyl groups, as a stored nonionic 15% microemulsion (supplier reference: Belsil ADM Log 1);
the polyether amines known notably under the reference Jeffamine from the company Huntsman; and notably: polyethylene glycol and/or polypropylene glycol α,ω-diamines (bearing a chain-end amine function), such as those sold under the names Jeffamine D-230, D-400, D-2000, D-4000, ED-600, ED-9000. ED-2003;
polytetrahydrofuran (or polytetramethylene glycol) α,ω-diamines, polybutadiene α,ω-diamines;
polyamidoamine (PANAM) dendrimers with amine end functions;
poly(meth)acrylates or poly(meth)acrylamides bearing primary or secondary amine side functions, such as poly(3-aminopropyl)methacrylamide or poly(2-aminoethyl) methacrylate.

As polyamine compounds bearing several amino polymer primary amine and/or secondary amine groups e), use is preferably made of polydi(C₁-C₄)alkylsiloxanes comprising primary amine groups at the chain end or on side chains.

Advantageously, the polyamine compounds e) used in the process according to the invention are chosen from polydi(C₁-C₄)alkylsiloxanes comprising primary amine groups at the chain end and/or on side chains.

More preferentially, the amine compound(s) iv) contained in composition (B) of the process of the invention are chosen from those of formulae (IVb) and (IVe) as defined previously and even more preferentially (IV′b) and (IV′e) as defined previously.

According to a particular embodiment of the invention, composition (B) also comprises one or more hydrocarbon-based oils iii) as defined previously. Advantageously, the hydrocarbon-based oils contained in dispersion (A) and in composition (B) are identical.

According to a preferred embodiment of the invention, the hydrocarbon-based oil(s) of composition (B) are chosen from hydrocarbon-based oils containing from 8 to 14 carbon atoms, in particular the apolar oils described previously. Even more preferentially, the hydrocarbon-based oil(s) of composition (B) are isododecane.

According to a particular embodiment of the invention, composition (B) is aqueous or aqueous-alcoholic. The term “aqueous-alcoholic” refers to a mixture of water and of a linear or branched C₂-C₄ alkanol, preferably ethanol.

According to one advantageous embodiment of the invention, composition (B) is aqueous or aqueous-alcoholic and preferably also comprises one or more chitosans and/or one or more polyamino acids, preferably polylysines.

According to another advantageous embodiment of the invention, composition (B) is aqueous and comprises one or more alkoxysilanes f) as defined previously in emulsion in water, in particular of water-in-oil (W/O) type.

According to yet another advantageous embodiment of the invention, composition (B) is aqueous and comprises one or more polyamines e) as defined previously in emulsion of oil-in-water (O/W) type.

Advantageously, the amine compound(s) used in the process according to the invention are used in a mole ratio of amine group of the amine compound iv)/ethylenically unsaturated anhydride compound of the anhydride b) as defined previously ranging from 0.01 to 10, preferably ranging from 0.1 to 5, preferentially ranging from 0, 1 to 2 and more preferentially ranging from 0.1 to 1.

The at least two-step process of the invention makes it possible to obtain deposits of cosmetic agents and in particular of pigments which have good resistance to the external attacking factors to which keratin materials may be subjected, notably good resistance to water and soap, shower gels and food oils.

In addition, the process of the invention makes it possible to trap nonvolatile compounds, notably oils, in order, for example, to improve the cosmeticity, or to give sheen to keratin materials, preferably the skin, notably facial skin such as the lips, and to do so in a manner that is resistant to external attacking factors.

Nonvolatile oils that may be mentioned include: hydrocarbon-based oils of animal origin such as perhydrosqualene; hydrocarbon-based plant oils such as liquid triglycerides of fatty acids of 4 to 10 carbon atoms, such as heptanoic or octanoic acid triglycerides, or alternatively sunflower oil, corn oil, soybean oil, grapeseed oil, sesame seed oil, apricot oil, macadamia oil, castor oil, avocado oil, caprylic/capric acid triglycerides, jojoba oil, shea butter oil; linear or branched hydrocarbons, of mineral or synthetic origin such as liquid paraffins and derivatives thereof, petroleum jelly, polydecenes, hydrogenated polyisobutene such as Parleam; synthetic esters and ethers, notably of fatty acids, for instance purcellin oil, isopropyl myristate, 2-ethylhexyl palmitate, 2-octyldodecyl stearate, 2-octyldodecyl erucate, isostearyl isostearate; hydroxylated esters, for instance isostearyl lactate, octyl hydroxystearate, octyidodecyl hydroxystearate, diisostearyl malate, triisocetyl citrate, fatty alkyl heptanoates, octanoates or decanoates; polyol esters, for instance propylene glycol dioctanoate, neopentyl glycol diheptanoate, diethylene glycol diisononanoate; and pentaerythritol esters: fatty alcohols containing from 12 to 26 carbon atoms, for instance octyldodecanol, 2-butyloctanol, 2-hexyldecanol, 2-undecylpentadecanol, oleyl alcohol; partially hydrocarbon-based and/or silicone-based fluoro oils; silicone oils, for instance volatile or nonvolatile, linear or cyclic polymethylsiloxanes (PDMS), which are liquid or pasty at room temperature, for instance cyclomethicones or dimethicones, optionally including a phenyl group, for instance phenyl trimethicones, phenytrimethyldiphenylsiloxanes, diphenylmethyldimethyltrisiloxanes, diphenyl dimethicones, phenyl dimethicones, polymethylphenylsiloxanes; mixtures thereof.

These oils may be present in a content ranging from 0.01% to 60% by weight and better still from 0.1% to 50% by weight relative to the total weight of dispersion (A).

Dispersion (A) and compositions (B) and (C) according to the invention may also comprise one or more dyestuffs chosen from liposoluble dyes and pulverulent dyestuffs, for instance the pigments, nacres and glitter flakes that are well known to those skilled in the art. The dyestuffs may be present in the composition in a content ranging from 0% to 30% by weight, relative to the weight of the dispersion or composition which comprises them, preferably from 0% to 10% by weight.

Dispersion (A) and compositions (B) and (C) according to the invention may also comprise one or more fillers, notably in a content ranging from 0.01% to 30% by weight, relative to the weight of the dispersion or composition which comprises them.

v) The Cosmetic Active Agent(s)

The cosmetic active agent(s) of the invention are chosen from a) pigments, b) active agents for caring for keratin materials, preferably the skin, and c) UV-screening agents, and also d) mixtures thereof.

According to a preferred embodiment of the present patent application, the cosmetic active agent(s) of the invention are chosen from a) pigments.

According to a particular embodiment of the present patent application, the cosmetic active agent(s) of the invention are chosen from b) active agents for caring for keratin materials, preferably skincare active agents.

According to another particular embodiment of the present patent application, the cosmetic active agent(s) of the invention are chosen from c) UV-screening agents.

According to a particular embodiment of the invention, dispersion (A) and/or composition (B) and/or composition (C) of the invention comprise v) one or more cosmetic active agents.

According to a particular embodiment of the invention, dispersion (A) comprises v) one or more cosmetic active agents, preferably one or more pigments.

According to another particular embodiment of the invention, composition (B) comprises v) one or more cosmetic active agents, preferably one or more pigments.

According to a preferred embodiment of the invention, composition (C) comprises v) one or more cosmetic active agents, preferably one or more pigments.

The hair dye(s) more particularly represent from 0.001% to 10% by weight and preferably from 0.005% to 5% by weight relative to the total weight of the composition which comprises them.

According to a particular embodiment of the invention, dispersion (A) comprises v) one or more cosmetic active agents, in particular one or more pigments.

According to another particular embodiment of the invention, composition (B) comprises v) one or more cosmetic active agents, in particular one or more pigments.

According to yet another preferred embodiment of the invention, composition (C) comprises v) one or more cosmetic active agents, in particular one or more pigments.

The term “pigment” refers to any pigment, of synthetic or natural origin, which gives color to keratin materials. The solubility of the pigments in water at 25° C. and at atmospheric pressure (760 mmHg) is less than 0.05% by weight, and preferably less than 0.01%.

They are white or colored solid particles which are naturally insoluble in the hydrophilic and lipophilic liquid phases usually employed in cosmetics or which are rendered insoluble by formulation in the form of a lake, where appropriate. More particularly, the pigments have little or no solubility in aqueous-alcoholic media.

The pigments that may be used are notably chosen from the organic and/or mineral pigments known in the art, notably those described in Kirk-Othmer's Encyclopedia of Chemical Technology and in Ullmann's Encyclopedia of Industrial Chemistry. Pigments that may notably be mentioned include organic and mineral pigments such as those defined and described in Ulmann's Encyclopedia of Industrial Chemistry “Pigments, organic”, 2005 Wiley-VCH Verlag GmbH & Co. KGaA, Weinheim 10.1002/14356007.a20 371 and ibid, “Pigments. Inorganic, 1. General”, 2009, Wiley-VCH Verlag GmbH & Co. KGaA, Weinheim10.1002/14356007.a20_243.pub3,

These pigments may be in pigment powder or paste form. They may be coated or uncoated. The pigments may be chosen, for example, from mineral pigments, organic pigments, lakes, pigments with special effects such as nacres or glitter flakes, and mixtures thereof.

The pigment may be a mineral pigment. The term “mineral pigment” refers to any pigment that satisfies the definition in Ullmann's encyclopedia in the chapter on inorganic pigments. Among the mineral pigments that are useful in the present invention, mention may be made of iron oxides, chromium oxides, manganese violet, ultramarine blue, chromium hydrate, ferric blue and titanium oxide.

The pigment may be an organic pigment. The term “organic pigment” refers to any pigment that satisfies the definition in Ullmann's encyclopedia in the chapter on organic pigments. The organic pigment may notably be chosen from nitroso, nitro, azo, xanthene, quinoline, anthraquinone, phthakocyanine, metal complex type, isoindolinone, isoindoline, quinacridone, perinone, perylene, diketopyrrolopyrrole, thioindigo, dioxazine, triphenylmethane and quinophthalone compounds.

In particular, the white or colored organic pigments may be chosen from carmine, carbon black, aniline black, azo yellow, quinacridone, phthaiocyanine blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified in the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerization of indole or phenolic derivatives as described in patent FR 2 679 771.

According to a particular embodiment of the invention, the pigment(s) used are pigment pastes of organic pigments such as the products sold by the company Hoechst under the name:
Cosmenyl Yellow 10G: Yellow 3 pigment (CI 11710):
Cosmenyl Yellow G: Yellow 1 pigment (CI 11680);
Cosmenyl Orange GR: Orange 43 pigment (CI 71105);
Cosmenyl Red R: Red 4 pigment (CI 12085);
Cosmenyl Carmine FB: Red 5 pigment (CI 12490);
Cosmenyl Violet RL: Violet 23 pigment (CI 51319);
Cosmenyl Blue A2R: Blue 15.1 pigment (CI 74160);
Cosmenyl Green GG: Green 7 pigment (CI 74260);
Cosmenyl Black R: Black 7 pigment (CI 77266).

The pigments in accordance with the invention may also be in the form of composite pigments, as described in patent EP 1 184 426. These composite pigments may be composed notably of particles including:
a mineral core,
at least one binder for fixing the organic pigments to the core, and
at least one organic pigment at least partially covering the core.

The term “lake” refers to dyes adsorbed onto insoluble particles, the assembly thus obtained remaining insoluble during use. The mineral substrates onto which the dyes are adsorbed are, for example, alumina, silica, calcium sodium borosilicate or calcium aluminum borosilicate and aluminum. Among the organic dyes, mention may be made of cochineal carmine.

Mention may be made, as examples of lakes, of the products known under the following names: D & C Red 21 (CI 45 380), D & C Orange 5 (CI 45 370). D & C Red 27 (CI 45 410), D & C Orange 10 (CI 45 425), D & C Red 3 (CI 45 430), D & C Red 7 (CI 15 850:1), D & C Red 4 (CI 15 510), D & C Red 33 (CI 17 200). D & C Yellow 5 (CI 19 140), D & C Yellow 6 (CI 15 985), D & C Green (CI 61 570), D & C Yellow 10 (CI 77 002), D & C Green 3 (CI 42 053) or D & C Blue 1 (CI 42 090).

The mineral substrates onto which the dyes are adsorbed are, for example, alumina, silica, calcium sodium borosilicate or calcium aluminum borosilicate and aluminum.

Among the dyes, mention may be made of cochineal carmine. Mention may also be made of the dyes known under the following names: D & C Red 21 (CI 45 380), D & C Orange 5 (CI 45 370). D & C Red 27 (CI 45 410). D & C Orange 10 (CI 45 425), D & C Red 3 (CI 45 430), D & C Red 4 (CI 15 510), D & C Red 33 (CI 17 200). D & C Yellow 5 (CI 19 140), D & C Yellow 6 (CI 15 985), D & C Green (CI 61 570), D & C Yellow 10 (CI 77 002), D & C Green 3 (CI 42 053). D & C Blue 1 (CI 42 090).

An example of a lake that may be mentioned is the product known under the following name: D&C Red 7 (CI 15 850:1).

The pigment(s) may also be pigments with special effects.

The term “pigments with special effects” refers to pigments that generally create a colored appearance (characterized by a certain shade, a certain vivacity and a certain level of luminance) that is nonuniform and that changes as a function of the conditions of observation (light, temperature, angles of observation, etc.). They thereby differ from colored pigments, which afford a standard uniform opaque, semitransparent or transparent shade.

Several types of pigments with special effects exist: those with a low refractive index, such as fluorescent, photochromic or thermochromic pigments, and those with a higher refractive index, such as nacres or glitter flakes.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with an organic dye notably of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or tint.

As illustrations of nacres that may be used in the context of the present invention, mention may notably be made of gold-colored nacres sold notably by the company Engelhard under the name Gold 222C (Cloisonne), Sparkle gold (Timica), Gold 4504 (Chromalite) and Monarch gold 233X (Cloisonne); the bronze nacres sold notably by the company Merck under the names Bronze fine (17384) (Colorona) and Bronze (17353) (Colorona), by the company Eckart under the name Prestige Bronze and by the company Engelhard under the name Super bronze (Cloisonne); the orange nacres sold notably by the company Engelhard under the names Orange 363C (Cloisonne) and Orange MCR 101 (Cosmica) and by the company Merck under the names Passion orange (Colorona) and Matte orange (17449) (Microna); the brown-tinted nacres sold notably by the company Engelhard under the names Nu-antique copper 340XB (Cloisonne) and Brown CL4509 (Chromalite): the nacres with a copper tint sold notably by the company Engelhard under the name Copper 340A (Timica) and by the company Eckart under the name Prestige Copper; the nacres with a red tint sold notably by the company Merck under the name Sienna fine (17386) (Colorona); the nacres with a yellow tint sold notably by the company Engelhard under the name Yellow (4502) (Chromalite); the red-tinted nacres with a golden tint sold notably by the company Engelhard under the name Sunstone G012 (Gemtone); the black nacres with a golden tint sold notably by the company Engelhard under the name Nu-antique bronze 240 AB (Timica); the blue nacres sold notably by the company Merck under the name Matte blue (17433) (Microna), Dark Blue (117324) (Colorona); the white nacres with a silvery tint sold notably by the company Merck under the name Xirona Silver; and the golden-green pinkish-orange nacres sold notably by the company Merck under the name Indian summer (Xirona), and mixtures thereof.

In addition to nacres on a mica support, multilayer pigments based on synthetic substrates such as alumina, silica, sodium calcium borosilicate or calcium aluminum borosilicate, and aluminum, may be envisaged.

Mention may also be made of pigments with an interference effect which are not attached to a substrate, such as liquid crystals (Helicones HC from Wacker) or interference holographic glitter flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

The variety of pigments that may be used in the present invention makes it possible to obtain a wide range of colors, and also particular optical effects such as metallic effects or interference effects.

The size of the pigment used in the cosmetic composition according to the present invention is generally between 10 nm and 200 μm, preferably between 20 nm and 80 μm and more preferably between 30 nm and 50 μm.

The pigments may be dispersed in the product by means of a dispersant.

The term “dispersant” refers to a compound which can protect the dispersed particles from agglomerating or flocculating. This dispersant may be a surfactant, an oligomer, a polymer or a mixture of several thereof, bearing one or more functionalities with strong affinity for the surface of the particles to be dispersed. In particular, they may become physically or chemically attached to the surface of the pigments. These dispersants also contain at least one functional group that is compatible with or soluble in the continuous medium. Said agent may be charged: it may be anionic, cationic, zwitterionic or neutral.

According to a particular embodiment of the invention, the dispersants used are chosen from 12-hydroxystearic acid esters, more particularly, and from C₈ to C₂₀ fatty acid esters of polyols such as glycerol or diglycerol, such as poly(12-hydroxystearic acid) stearate with a molecular weight of approximately 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Unigema, and mixtures thereof.

As other dispersants that may be used in the compositions of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The pigments used in the cosmetic composition according to the invention may be surface-treated with an organic agent.

Thus, the pigments surface-treated beforehand that are useful in the context of the invention are pigments which have been completely or partially subjected to a surface treatment of chemical, electronic, electrochemical, mechanochemical or mechanical nature with an organic agent, such as those notably described in Cosmetics and Toiletries. February 1990, Vol. 105, pages 53-64, before being dispersed in the composition in accordance with the invention. These organic agents may be chosen, for example, from amino acids; waxes, for example camauba wax and beeswax; fatty acids, fatty alcohols and derivatives thereof, such as stearic acid, hydroxystearic acid, stearyl alcohol, hydroxystearyl alcohol and lauric acid and derivatives thereof; anionic surfactants; lecithins; sodium, potassium, magnesium, iron, titanium, zinc or aluminum salts of fatty acids, for example aluminum stearate or laurate; metal alkoxides; polysaccharides, for example chitosan, cellulose and derivatives thereof; polyethylene: (meth)acrylic polymers, for example polymethyl methacrylates; polymers and copolymers containing acrylate units; proteins; alkanolamines; silicone compounds, for example silicones, polydimethylsiloxanes, alkoxysilanes, alkylsilanes and siloxysilicates; organofluorine compounds, for example perfluoroalkyl ethers; fluorosilicone compounds.

The surface-treated pigments that are useful in the cosmetic composition according to the invention may also have been treated with a mixture of these compounds and/or may have undergone several surface treatments.

The surface-treated pigments that are useful in the context of the present invention may be prepared according to surface-treatment techniques that are well known to those skilled in the art, or may be commercially available as is.

Preferably, the surface-treated pigments are coated with an organic layer.

The organic agent with which the pigments are treated may be deposited on the pigments by evaporation of solvent, chemical reaction between the molecules of the surface agent or creation of a covalent bond between the surface agent and the pigments.

The surface treatment may thus be performed, for example, by chemical reaction of a surface agent with the surface of the pigments and creation of a covalent bond between the surface agent and the pigments or the fillers. This method is notably described in patent U.S. Pat. No. 4,578,266.

An organic agent covalently bonded to the pigments will preferably be used.

The agent for the surface treatment may represent from 0.1% to 50% by weight, preferably from 0.5% to 30% by weight and even more preferentially from 1% to 10% by weight relative to the total weight of the surface-treated pigments.

Preferably, the surface treatments of the pigments are chosen from the following treatments:
a PEG-silicone treatment, for instance the AQ surface treatment sold by LCW;
a chitosan treatment, for instance the CTS surface treatment sold by LCW;
a triethoxycaprylylsilane treatment, for instance the AS surface treatment sold by LCW;
a methicone treatment, for instance the SI surface treatment sold by LCW;
a dimethicone treatment, for instance the Covasil 3.05 surface treatment sold by LCW;
a dimethicone/trimethyl siloxysilicate treatment, for instance the Covasil 4.05 surface treatment sold by LCW;
a lauroyllysine treatment, for instance the LL surface treatment sold by LCW;
a lauroyllysine dimethicone treatment, for instance the LL/SI surface treatment sold by LCW;
a magnesium myristate treatment, for instance the MM surface treatment sold by LCW;
an aluminum dimyristate treatment, such as the MI surface treatment sold by Miyoshi;
a perfluoropolymethyl isopropyl ether treatment, for instance the FHC surface treatment sold by LCW;
an isostearyl sebacate treatment, for instance the HS surface treatment sold by Miyoshi;
a disodium stearoyl glutamate treatment, for instance the NAI surface treatment sold by Miyoshi:
a dimethicone/disodium stearoyl glutamate treatment, for instance the SA/NAI surface treatment sold by Miyoshi;
a perfluoroalkyl phosphate treatment, for instance the PF surface treatment sold by Daito;
an acrylate/dimethicone copolymer and perfluoroalkyl phosphate treatment, for instance the FSA surface treatment sold by Daito;
a polymethylhydrosiloxane/perfluoroalkyl phosphate treatment, for instance the FS01 surface treatment sold by Daito;
a lauryllysine/aluminum tristearate treatment, for instance the LL-StAI surface treatment sold by Daito;
an octyltriethylsilane treatment, for instance the OTS surface treatment sold by Daito;
an octyltriethylsilane/perfluoroalkyl phosphate treatment, for instance the FOTS surface treatment sold by Daito;
an acrylate/dimethicone copolymer treatment, for instance the ASC surface treatment sold by Daito;
an isopropyl titanium triisostearate treatment, for instance the ITT surface treatment sold by Daito;
a microcrystalline cellulose and carboxymethylcellulose treatment, for instance the AC surface treatment sold by Daito;
a cellulose treatment, for instance the C2 surface treatment sold by Daito;
an acrylate copolymer treatment, for instance the APD surface treatment sold by Daito;
a perfluoroalkyl phosphate/isopropyl titanium trdisostearate treatment, for instance the PF+ITT surface treatment sold by Daito.
The composition in accordance with the present invention may furthermore comprise one or more surface-untreated pigments.
According to a particular embodiment of the invention, the pigment(s) are mineral pigments.
According to another particular embodiment of the invention, the pigment(s) are chosen from nacres.

According to a particular embodiment of the invention, the dispersant is present with organic pigments in dispersion (A), and/or composition (B) and/or (C) or with inorganic pigments in particulate form of submicron size.

The term “submicron” or “submicronic” refers to pigments having a particle size that has been micronized by a micronization method and having a mean particle size of less than a micrometer (μm), in particular between 0.1 and 0.9 μm, and preferably between 0.2 and 0.6 μm.

According to one embodiment, the dispersant and the pigment(s) are present in an amount (dispersant:pigment) of between 0.5:1 and 2:1, particularly between 0.75:1 and 1.5:1 or better still between 0.8:1 and 1.2:1.

According to a particular embodiment, the dispersant is suitable for dispersing the pigments and is compatible with a condensation-curable formulation.

The term “compatible” means, for example, that said dispersant is miscible in the oily phase of the composition or of the dispersion containing the pigment(s), and it does not retard or reduce the curing. The dispersant is preferably cationic.

The dispersant(s) may therefore have a silicone backbone, such as silicone polyether and dispersants of amino silicone type. Among the suitable dispersants that may be mentioned are:
amino silicones, i.e. silicones comprising one or more amino groups such as those sold under the names and references: BYK LPX 21879 by BYK, GP-4. GP-6, GP-344. GP-851. GP-965. GP-967 and GP-988-1, sold by Genesee Polymers,
silicone acrylates such as Tego® RC 902, Tego RC 922, Tego® RC 1041, and Tego® RC 1043, sold by Evonik,
polydimethylsiloxane (PDMS) silicones bearing carboxylic groups, such as X-22162 and X-22370 by Shin-Etsu, epoxy silicones such as GP-29, GP-32, GP-502, GP-504, GP-514, GP-607, GP-682, and GP-695 by Genesee Polymers, or Tegop RC 1401, Tegof RC 1403, Tego RC 1412 by Evonik.

According to a particular embodiment, the dispersant(s) are of aminosilicone type and are positively charged.

Mention may also be made of dispersants bearing chemical groups that are capable of reacting with the reagents of the oily phase and are thus capable of improving the 3D network formed from the aminosilicones. For example, dispersants of epoxy silicone pigments can react chemically with the amino silicone prepolymer amino group(s) to increase the cohesion of the amino silicone film comprising the pigment(s).

Preferably, the pigment(s) v) of the invention are chosen from carbon black, iron oxides, notably black iron oxides, and micas coated with iron oxide, triarylmethane pigments, notably blue and violet triarylmethane pigments, such as Blue 1 Lake, azo pigments, notably red azo pigments, such as D&C Red 7, an alkali metal salt of lithol red, such as the calcium salt of lithol red B.

According to a particular embodiment of the invention, the amount of pigments ranges from 0.5% to 40% and preferably from 1% to 20% relative to the weight of the composition and dispersion comprising them.

vi) The Plasticizer(s)

According to a particular embodiment, the process uses one or more plasticizers. Said plasticizer(s) may be present in composition (A) and/or (B) and/or (C).

The term “plasticizer” or “plasticizing agent” means an organic chemical compound, which is in solid state or in liquid state, at room temperature and at atmospheric pressure, and which is added to a composition comprising different types of polymerizable ingredients (monomers) to make the polymer more supple, more flexible, and/or to improve its mechanical strength. Plasticizers are known to those skilled in the art; mention may be made, for example, of “Plasticizers”, Encyclopedia of polymer Science and Technology, Helmut Reinecke, Rodrigo Navarro, Mónica Perez, https://doi.org/10.1002/0471440264.pst245.pub215 September 2011.

For the purposes of the invention, the plasticizers have a molecular weight of between 200 and 1000 g/mol, particularly between 300 and 700, preferably between 350 and 600. The agents are also organic compounds consisting of carbon and hydrogen atoms and of one or more heteroatoms chosen from oxygen, sulfur and silicon atoms, in particular chosen from oxygen and silicon atoms, preferably at least 3 heteroatoms, even more preferentially between 4 and 10 heteroatoms, and which may contain one or more aryl groups such as benzyl. In particular, they comprise one or more groups chosen from esters, phthalate, benzoate, sulfonate, citrates and siloxanes.

Preferably, the plasticizer(s) of the invention are chosen from those of the phthalate, ester, citrate, benzoate, citrate and siloxane families.

According to a particular embodiment of the invention, the plasticizer(s) are chosen from compounds (V) and (Vi) below:

R^{h}—O—C(O)-L-[C(O)-Q-Rⁱ]ₙ (V)

in which formula (V):
R^{h} and Rⁱ, which may be identical or different, represent a group from among: (C₁-C₂₀)alkyl, aryl such as phenyl, aryl(C₂-C₄)alkyl such as benzyl, preferably (C₁-C₄)alkyl such as n-butyl;
n is 1, 2 or 3, preferably 2; and
L represents a group from among: a) divalent, trivalent or tetravalent C₁-C₁₀ alkyl, preferably trivalent (C₂-C₈)alkyl, said divalent, trivalent or tetravalent alkyl possibly being optionally substituted with one or more hydroxyl groups, b) divalent or trivalent cycloalkyl, or c) divalent, trivalent or tetravalent aryl, preferably divalent or trivalent phenyl; preferably, L represents a group a);

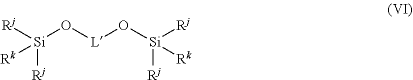

in which formula (VI):
Rⁱ, which may be identical or different, preferably identical, represents a group from among: (C₁-C₁₀)alkyl, aryl such as phenyl, aryl(C₁-C₄)alkyl such as benzyl, preferably aryl such as phenyl,
Rⁱ, which may be identical or different, preferably identical, represents a hydrogen atom or a hydroxyl, (C₁-C₆)alkyl or (C₁-C₆)alkoxy group,
L′ represents a group chosen from a) divalent C₂-C₁₀ alkyl, preferably divalent (C₄-C₈)alkyl, which may be optionally substituted with one or more hydroxyl groups, and/or interrupted with one or more oxygen atoms, and/or interrupted with one or more divalent groups —Si(Rⱼ)(R^{k})— with R^{j} and R^{k} as defined previously, b) divalent or trivalent cycloalkyl, c) divalent, trivalent or tetravalent aryl, and d) —Si(R^{j})(R^{k})—; preferably, L′ represents a group d) —Si(R^{j})(R^{k})—.

According to a particular embodiment of the invention, the compounds of formula (V) are chosen from the compounds of formula (V′):

R^{h}—O—C(O)-L-[C(O)—O—Rⁱ]^{c} (V′)

in which formula (V):
R^{h} and Rⁱ, which may be identical or different, represent a group from among: (C₁-C₆)alkyl, aryl(C₁-C₄)alkyl such as benzyl, preferably (C₁-C₄)alkyl such as n-butyl;
n is 1 or 2, preferably 2; and
L represents a group from among: a) divalent, trivalent or tetravalent C₂-C₆ alkyl, preferably trivalent (C₂-Cₑ)alkyl, said alkyl possibly being optionally substituted with one or more hydroxyl groups, preferably substituted with one hydroxyl group.

According to a particular embodiment of the invention, the compounds of formula (VI) are chosen from the compounds of formula (VI′):

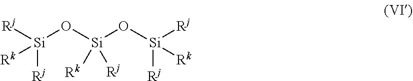

in which formula (VI′):
R^{j}, which may be identical or different, preferably identical, represents a group from among: aryl such as phenyl, aryl(C₁-C₄)alkyl such as benzyl, preferably aryl such as phenyl, and
R^{j}, which may be identical or different, preferably identical, represents a (C₁-C₆)alkyl and preferably (C₁-C₄)alkyl group such as methyl.

In particular, the plasticizer(s) of the invention are chosen from: di-n-hexyl phthalate (DnHP), diisoheptyl phthalate, (DIHP), diheptyl phthalate (DnHP), bis(2-ethylhexyl) phthalate (DEHP), diheptylnonyl phthalate (DnHNP), di-n-octyldecyl phthalate (DNODP), diheptyinonylundecyl phthalate (DnHNUP), dilsononyl phthalate (DINP), dinonyl phthalate (DNP), di-n-nonyl phthalate (DnNP) diisodecyl phthalate (DIDP), di-n-nonyldecylundecyl phthalate (DnNDUP), dinonylundecyl phthalate (DnNUP), diundecyl phthalate (DUP), diisoundecyidodecyl phthalate (UDP), ditridecyl phthalate (DTDP), bis(2-ethylhexyl) terephthalate (DOTP), butylbenzyl phthalate (BBP), diheptylnonyl adipate (DnHNA), bis(2-ethylhexyl) adipate (DEHA), diisononyl adipate (DINA), diisodecyl adipate (DIDA), triheptyinonyl trimellitate (TnHNTM), tris(2-ethythexyl) trimellitate (TOTM), triisononyl trimellitate (TINTM), bis(2-ethylhexyl) sebacate (DOS), bis(2-ethylhexyl) azelate (DOZ), tributyl citrate and trimethylpentaphenyltrisiloxane; preferably, the plasticizer(s) of the invention are chosen from: tri(C₁-C₆)alkyl citrates such as tributyl citrate and trimethylpentaphenyltrisiloxane.

The plasticizer(s) vi) of the invention may be present in composition (A) and/or in composition (B), and/or in another composition (C). Preferably, the plasticizer(s) of the invention are present in composition (A) or (C), preferably (A).

According to a particular embodiment of the invention, the plasticizer(s) are present in an amount of between 5% and 50% by weight relative to the total weight of the polymer(s) a) to d) of the particles.

According to a particular embodiment of the invention, the plasticizers are present in an amount of between 0.5% and 30% by weight, in particular between 1% and 20%, preferably from 1.5% to 10% by weight relative to the total weight of composition (A), and/or in composition (8), and/or in another composition (C), preferably relative to the total weight of composition (A).

The Process of the Invention:

According to a particular embodiment of the invention, the process is a process for treating keratin materials, in particular the skin, preferably human skin and more preferentially facial skin such as the lips, comprising:
1) the application to said materials of an oily dispersion (A), which is preferably anhydrous, comprising:
   i) one or more particles consisting of one or more ethylenic copolymers:
      a) of (C₁-C₄)alkyl (C₁-C₄)(alkyl)acrylate, preferably (C₁-C₄)alkyl (meth)acrylate; and
      b) of ethylenically unsaturated anhydride compound, preferably maleic anhydride; and
   ii) one or more stabilizers consisting of ethylenic polymers chosen from:
      c) polymers of (C₃-C₁₂)cycloalkyl (C₁-C₆)(alkyl)acrylate monomers; and
      d) copolymers of (C₃-C₁₂)cycloalkyl (C₁-C₆)(alkyl)acrylate and (C₁-C₄)alkyl (C₁-C₄)(alkyl)acrylate; and
      e) one or more hydrocarbon-based oil(s);
2) the application to said materials of a composition (B), which is preferably anhydrous, comprising:
   f) one or more amine compound(s) chosen from:
   g) polyamine compounds bearing several primary amine and/or secondary amine groups, and
   h) amino alkoxysilanes,

it being understood that:
the process of the invention uses v) one or more cosmetic active agents chosen from a) pigments, b) active agents for caring for keratin materials, preferably the skin, and c) UV-screening agents, and also d) mixtures thereof, ingredients a) to d) possibly being found in another composition (C); and
composition (A) is applied to said materials, and composition (B) is then applied to said materials, and composition (C) is then applied to said materials.

According to another particular embodiment of the invention, the process is a process for treating keratin materials, in particular the skin, preferably human skin and more preferentially facial skin such as the lips, using compositions (A). (B) and (C), in which:
the process of the invention uses v) one or more cosmetic active agents chosen from a) pigments, b) active agents for caring for keratin materials, preferably the skin, and c) UV-screening agents, and also d) mixtures thereof, ingredients a) to d), preferably a), being found in composition (C); and
composition (C) is applied to said materials, and composition (A) is then applied to said materials, and composition (B) is then applied to said materials.

According to yet another embodiment of the invention, the process is a process for treating keratin materials, in particular the skin, preferably human skin and more preferentially facial skin such as the lips, using compositions (A), (B) and (C), in which:
the process of the invention uses v) one or more cosmetic active agents chosen from a) pigments, b) active agents for caring for keratin materials, preferably the skin, and c) UV-screening agents, and also d) mixtures thereof, ingredients a) to d), preferably a), being found in composition (C); and
composition (C) is applied to said materials, and compositions (A) and (B) are then applied simultaneously to said materials, or else a mixture of compositions (A)+(B) is applied to said materials after the application of composition (C) to said materials.

At Least Two-Step Application Process:

According to an advantageous variant of the invention, the process of the invention is a process for treating keratin materials, in particular the skin, preferably human skin and more preferentially facial skin such as the lips, comprising:
1) the application to said materials of an oily dispersion (A) comprising:
   i) one or more particles constituted of one or more ethylenic copolymers a) and b) as defined previously; and
   ii) one or more stabilizers constituted of ethylenic polymers chosen from c) and d) as defined previously;
   iii) one or more hydrocarbon-based oils as defined previously, and v) one or more cosmetic agents, preferably pigments;
      followed by
2) the application to said materials of a composition (B) comprising:
   iv) one or more amine compounds chosen from e) and f) as defined previously.

According to another advantageous variant of the invention, the process of the invention is a process for treating keratin materials, in particular the skin, preferably human skin and more preferentially facial skin such as the lips, comprising:
1) the application to said materials of an oily dispersion (A) comprising:
   i) one or more particles constituted of one or more ethylenic copolymers a) and b) as defined previously; and
   ii) one or more stabilizers constituted of ethylenic polymers chosen from c) and d) as defined previously; and
   iii) one or more hydrocarbon-based oils as defined previously;
      followed by
2) the application to said materials of a composition (B) comprising:
   iv) one or more amine compounds chosen from e) and f) as defined previously; and
   v) one or more cosmetic agents, preferably pigments.

According to yet another variant of the invention, the process of the invention is a process for treating keratin materials, in particular the skin, preferably human skin and more preferentially facial skin such as the lips, comprising:
1) the application to said materials of an oily dispersion (A) comprising:
   i) one or more particles constituted of one or more ethylenic copolymers a) and b) as defined previously; and
   ii) one or more stabilizers constituted of ethylenic polymers chosen from c) and d) as defined previously; and
   iii) one or more hydrocarbon-based oils as defined previously;
      followed by
2) the application to said materials of a composition (B) comprising:
   iv) one or more amine compounds chosen from e) and f) as defined previously; followed by
3) the application to said materials of a composition (C) comprising:
   v) one or more cosmetic active agents, preferably pigments.

According to yet another advantageous variant of the invention, the process of the invention is a process for treating keratin materials, in particular the skin, preferably human skin and more preferentially facial skin such as the lips, comprising:
1) the application to said materials of an oily dispersion (A) comprising:
   i) one or more particles constituted of one or more ethylenic copolymers a) and b) as defined previously; and
   ii) one or more stabilizers constituted of ethylenic polymers chosen from c) and d) as defined previously; and
   iii) one or more hydrocarbon-based oils as defined previously;
      followed by
2) the application to said materials of a composition (C) comprising v) one or more cosmetic active agents, preferably pigments; followed by
3) the application to said materials of a composition (8) comprising iv) one or more amine compounds chosen from e) and f) as defined previously.

According to a particularly preferred variant of the invention, the process of the invention is a process for treating keratin materials, in particular the skin, preferably human skin and more preferentially facial skin such as the lips, comprising:
1) the application to said materials of a composition (C) comprising v) one or more cosmetic active agents, preferably pigments; followed by
2) the application to said materials of an oily dispersion (A) comprising:
   i) one or more particles constituted of one or more ethylenic copolymers a) and b) as defined previously; and
   ii) one or more stabilizers constituted of ethylenic polymers chosen from c) and d) as defined previously; and
   iii) one or more hydrocarbon-based oils as defined previously;
      followed by
3) the application to said materials of a composition (B) comprising iv) one or more amine compounds chosen from e) and f) as defined previously.

According to another particular variant of the invention, the cosmetic agent(s) v) as defined previously, in particular the pigments, are in a composition (C). Said composition may be applied simultaneously with composition (A), or with composition (B). Preferably, composition (C) is applied after step 1), i.e. after the application of composition (A); more preferentially, compositions (A) and (B) do not comprise any hair dye and a composition (C) comprising one or more hair dyes v) as defined previously is applied after step 1) of the process of the invention, followed by the application to the keratin materials of composition (B).

The dispersion of the invention, dispersion (A), and compositions (B) and (C) are cosmetic, i.e. they comprise only cosmetically acceptable ingredients.

According to a preferred embodiment of the invention, dispersion (A) and compositions (B) and (C) are anhydrous.

According to another advantageous embodiment, dispersion (A) and composition (8) are anhydrous, and composition (C) is aqueous.

According to a particular embodiment of the invention, composition (B) is aqueous or aqueous-alcoholic.

According to another particularly advantageous embodiment of the invention, composition (C) is aqueous or aqueous-alcoholic.

According to yet another advantageous embodiment, dispersion (A) is anhydrous and composition (B) and composition (C) are aqueous or aqueous-alcoholic, preferably aqueous.

Compositions (B) and (C) and dispersion (A) according to the invention may comprise a cosmetic additive chosen from water, fragrances, preserving agents, fillers, oils, waxes, surfactants, moisturizers, vitamins, ceramides, antioxidants, free-radical scavengers, polymers, thickeners and dyestuffs.

Compositions (B) and (C) and dispersion (A) according to the invention may also comprise other dyestuffs, such as liposoluble dyes or water-soluble dyes. This dyestuff may be present in a content ranging from 0.01% to 30% by weight, relative to the total weight of the composition containing them, preferably from 0.01% to 10% by weight.

The liposoluble dyes are, for example, Sudan Red, D&C Red 17. D&C Green 6, p-carotene, soybean oil, Sudan Brown, D&C Yellow 11, D&C Violet 2, D&C Orange 5, quinoline yellow and annatto. The water-soluble dyes are, for example, beetroot juice or methylene blue.

According to one embodiment, dispersion (A) and compositions (B) and (C) according to the invention are anhydrous compositions.

Preferentially, the first step of the process of the invention is the application of dispersion (A) in one or more volatile apolar solvents, notably isododecane.

According to another variant, step 2) follows step 1) without intermediate rinsing or drying. Preferably, after applying dispersion (A) during step 1), there is a waiting time of between 1 minute and 6 hours, in particular between 10 minutes and 5 hours, more particularly between 30 minutes and 4 hours, and preferably about 3 hours, before applying composition (B).

If the first step is the step of applying composition (C) followed by the application of the oily dispersion (A) and then the application of composition (A) between the application of composition (C) (step 1) and the step of applying dispersion (A) (step 2), the keratin materials are preferably dried naturally.

If the first step is the step of applying the oily dispersion (A) and the second step is that of applying composition (C) and the third step is that of applying composition (B) between the application of composition (C) as defined previously (step 2) and the step of applying composition (B) (step 3), the keratin materials are preferably dried naturally.

If the first step is the application of the oily dispersion (A) followed by the application of composition (B) and then the application of composition (C) between the application of composition (C) (step 3) and the step of applying composition (B) (step 2), the keratin materials are preferably dried naturally.

The Kit

A subject of the invention is also a kit or device with several separate compartments comprising:
in a first compartment: dispersion (A) as defined previously,
in a second separate compartment: composition (B) as defined previously, and
optionally in a third compartment separate from the other two: composition (C) as defined previously.

The composition packaging assembly is, in a known manner, any packaging that is suitable for storing cosmetic compositions (notably a bottle, tube, spray bottle or aerosol can).

A subject of the invention is also the oily dispersion (A) as defined previously, comprising v) the cosmetic agent(s) as defined previously, it being understood that when v) represents one or more pigment(s), then the dispersion is anhydrous and does not comprise any polyamine compound bearing several primary amine and/or secondary amine groups and does not comprise any amino alkoxysilanes.

A subject of the invention is also the oily dispersion (A) which is in an inverse emulsion (W/O) as defined previously.

The invention is illustrated in greater detail in the examples that follow.

### EXAMPLES

### Example 1

The oily dispersions (A) are formed as a whole [particles i)+stabilizer ii)] containing:
70% by weight of (C₁-C₄)alkyl (C₁-C₄)(alkyl)acrylate a) (such as ethyl acrylate),
10% by weight of ethylenically unsaturated anhydride compound b) (such as maleic anhydride), and
20% by weight of polymers of (C₃-C₁₂)cycloalkyl (C₁-C₆)(alkyl)acrylate monomers c) (such as isobornyl acrylate).

The preparation of these oily dispersions was performed in a 1 liter pilot reactor. The synthesis is performed in two steps:

In a first step, isobornyl acrylate is polymerized in isododecane/ethyl acetate (60/40) in the presence of a small amount of ethyl acrylate and of a radical initiator (T21S). In the first step, the isobornyl acrylate/ethyl acrylate mass ratio is 92/8.

In the second step, the rest of the ethyl acrylate and the maleic anhydride are added in the presence of isododecane/ethyl acetate (60/40) and of the radical initiator Trigonox 21S (T21S).

After stripping, the polymer is at a solids content of 52% in the isododecane. The ratios employed to obtain the stabilizer and the particulate core are summarized in the table below:

| | Mass percentage | | Mass percentage in |
|---|---|---|---|
| | Stabilizer - | | the stabilizer and |
| | Particulate core | Monomer | the particulate core |
| Stabilizer ii) | 22 | Isobornyl acrylate | 92 |
| | | Ethyl acrylate | 8 |
| Particulate | 78 | Ethyl acrylate | 87 |
| core i) | | Maleic anhydride | 13 |

Amount of Reagents:

Step 1:

| Reagents: | Mass (g) |
|---|---|
| Isobornyl acrylate | 50 |
| Ethyl acrylate | 4 |
| T21S | 0.54 |
| Isododecane/EtOAc (60/40) | 98 |

Isododecane added between the two steps:

| Reagent | Mass (g) |
|---|---|
| Isododecane/EtOAc (60/40) | 80 |

Step 2:

| | | Mass added to the beaker |
|---|---|---|
| Reagents: | Mass (g) | for the addition (g) |
| Ethyl acrylate | 171 | 196.65 |
| Maleic anhydride | 25 | 28.7 |
| T21S | 1.96 | 2.25 |
| isododecane/EtOAc | 196 | 225.4 |
| (60/40) | | |

Experimental Protocol:

Isododecane/ethyl acetate (60/40), isobornyl acrylate, ethyl acrylate and T21S are introduced as feedstock into a reactor. The medium is heated to 90° C. under argon and with stirring. The solids content during this first step is 35.9%.

After heating for 2 hours, NMR indicates an isobornyl acrylate consumption of 97% (ethyl acrylate consumption: 97%).

After 2 hours of reaction, isododecane/ethyl acetate (60/40) are introduced into the feedstock. The medium is heated to 90° C.

Once the medium is at 90° C., ethyl acrylate/maleic anhydride, isododecane/ethyl acetate (60/40) and T21S are introduced over 2 hours by pouring. At the end of the introduction by pouring, the medium is milky. The solids content is 40%.

After 7 hours of synthesis, traces of the starting monomers remain.

1 L of isododecane and of ethyl acetate are then stripped out (NMR indicates that there are no more monomers and that the ethyl acetate has been totally removed from the dispersion). The solids content is about 52%.

### Example 2

The combination of particles i)+stabilizer ii) was prepared in the following manner:
75% by weight of (C₁-C₄)alkyl (C₁-C₄)(alkyl)acrylate a) (such as ethyl acrylate),
5% by weight of ethylenically unsaturated anhydride compound b) (such as maleic anhydride), and
20% by weight of polymers of (C₃-C₁₂)cycloalkyl (C₁-C₆)(alkyl)acrylate monomers c) (such as isobornyl acrylate).

The synthesis was performed in a 1 liter pilot reactor in two steps:

In a first step, isobornyl acrylate is polymerized in isododecane/ethyl acetate (60/40) in the presence of a small amount of ethyl acrylate and of a radical initiator (T21S). In the first step, the isobornyl acrylate/ethyl acrylate mass ratio is 92/8.

In the second step, the rest of the ethyl acrylate and the maleic anhydride are added in the presence of isododecane/ethyl acetate (60/40) and of the radical initiator Trigonox 21S (T21S).

After stripping, the polymer is at a solids content of 49% in the isododecane.

The ratios employed to obtain the stabilizer and the particulate core are summarized in the table below:

| | Mass percentage | | Mass percentage in |
|---|---|---|---|
| | Stabilizer - | | the stabilizer and |
| | Particulate core | Monomer | the particulate core |
| Stabilizer ii) | 22 | Isobornyl acrylate | 92 |
| | | Ethyl acryiate | 8 |
| Particulate | 78 | Ethyl acrylate | 94 |
| core i) | | Maleic anhydride | 6 |

Amount of Reagents:

Step 1:

| Reagents: | Mass (g) |
|---|---|
| Isobornyl acrylate | 50 |
| Ethyl acrylate | 4 |
| T21S | 0.52 |
| Isododecane/EtOAc (60/40) | 96 |

Isododecane added between the two steps:

| Reagent | Mass (g) |
|---|---|
| Isododecane/EtOAc (60/40) | 80 |

Step 2:

| | | Mass added to the beaker |
|---|---|---|
| Reagents: | Mass (g) | for the addition (g) |
| Ethyl acrylate | 183.5 | 211.03 |
| Maleic anhydride | 12.5 | 14.38 |
| T21S | 1.96 | 2.25 |
| Isododecane/EtOAc | 196 | 225.4 |
| (60/40) | | |

Experimental Protocol:

Isododecane/ethyl acetate (60/40), isobornyl acrylate, ethyl acrylate and T21S are introduced as feedstock into the reactor. The medium is heated to 90° C. (nominal medium temperature) under argon and with stirring. The solids content during this first step is 35.9%. After heating for 2 hours, NMR indicates an isobornyl acrylate consumption of 98% (ethyl acrylate consumption: 97%).

After 2 hours of reaction, isododecane/ethyl acetate (60/40) are introduced into the feedstock. The medium is heated to 90° C.

Once the medium is at 90° C., ethyl acrylate/maleic anhydride, isododecane/ethyl acetate (60/40) and T21S are introduced over 2 hours by pouring. At the end of the introduction by pouring, the medium is milky. The solids content is 40%.

After 7 hours of synthesis, traces of the starting monomers remain.

1 L of isododecane and of ethyl acetate are then stripped out (NMR indicates that there are no more monomers and that the ethyl acetate has been totally removed from the dispersion). The solids content is about 49%.

Amine Compounds Used:

| Amine compound 1 | Amine compound 2 |
|---|---|
| Poly(dimethylsiloxane),bis(3- | Bis-cetearyl amodimethicone, |
| aminopropyl) | 2-[(2-aminoethyl)amino]propyl Me, |
| terminated (PDMS-diNH₂) | di-Me, [(C₁₄-₂₀- |
| (CAS Number: 106214-84-0) | alkyldimethylsilyl)oxy]-terminated |
| (Mn = 50 000 g/mol) | (CAS Number: 1126942-72-0) |

Reaction of these amine compounds with the anhydride functions of the copolymer makes it possible to trap the nonvolatile oils and nevertheless to obtain deposits that do not transfer, that are not tacky and that are resistant to food oils and are glossy and comfortable.

Process for Application to Keratin Materials:

In a first stage, a formulation containing an oily dispersion of particles (A) was prepared (examples 1 and 2). The evaluations are performed on BioSkin or on a contrast card.

In this case, the contrast card reference is: Byk company—Product: Black scrub panels P121-10N, category number: 5015—dimension 165×432 mm. The formulations were then applied onto a glass support of the type such as byko-charts, black scrub panels from the company Byk and were left to dry for 24 hours.

In a first stage, a film of each formulation is deposited on a BioSkin sample by means of a film spreader. The BioSkin sample has the following approximate dimensions: (L: 10 cm; l: 6 cm; e: 2.5 mm). The thickness of the wet deposit is 50 μm. The films are dried for 24 hours at room temperature. The dry mass of formulation deposited on the BioSkin ranges between 60 and 100 mg. Once the films are dry, the tests may be performed.

After drying for 24 hours, formulation (B) containing an amine compound (compound 1 or 2) was applied.

After drying for 24 hours, evaluations of the deposits are performed:
Deposition of 0.5 mL of olive oil or of water over 5 minutes onto the deposit. After 5 minutes of contact, cotton wool is wiped over 15 times and the degradation of the deposit is observed*.
Feel of the deposit to observe the tacky nature and the transfer

For each combination, the evaluations were performed for composition (A) after drying and after application of composition (A) and (B) after drying.
0.5 mL of olive oil is applied to the film of formulation. After 5 minutes, the olive oil is removed by wiping 15 times with cotton wool. The deterioration of the film following contact with the olive oil and the transfer onto the cotton wool are thus examined.

If a deposit shows good resistance, the cotton wool remains white and the deposit has not degraded. Conversely, if substantial transfer is observed, the cotton wool is red and the film is completely degraded. The same protocol is applied with water

Thus, a grade is given for the resistance to olive oil/water but also for the transfer resistance ranging from:
very good resistance: ++ to poor resistance: −−
Transfer resistance: No transfer: ++ to substantial transfer: −−

Below are two examples of a good candidate for very good resistance to olive oil and for transfer resistance and of a poor candidate:

The evaluation and grading are performed in the following manner:
very good resistance: ++ to poor resistance: −−
Transfer resistance: No transfer: ++ to substantial transfer: −−

Fragmentation Test of the Formulations on BioSkin

As for the tests of resistance to olive oil and to Scotch, films are applied only on a sample of BioSkin. After drying for one day, the BioSkin plate is removed, by hand force, 10 times in the same manner.

The result may then be observed on the formulation film (fragmentation or otherwise).

In this case, a grade is given ranging from No fragmentation: ++ to substantial fragmentation: −−

Two response examples are presented below:

Protocol for Measuring the Gloss

Protocol for Measuring the Gloss

A film with a wet thickness of 50 μm is deposited on a contrast card.

The contrast card reference is: Byk company—Product: Byko-charts opacity 2A, category number: 2810—dimension 140×254 mm

The gloss is subsequently measured using a reference glossmeter: Lange company—Product: REFO 3D Portable glossmeter with three-angle geometry

### Example 1: Formula Containing Only Volatile Oils—Polymer of Example 1+Amine Compound 1

Composition (A1)

| Ingredients | Amount (g) |
|---|---|
| Combination of particle example 1, ingredients i), ii) | 25 (a.m.) |
| Pigment paste 40 wt % of DC Red 7 in isododecane, | 5 |
| ingredient v) | |
| Isododecane, ingredient iii) | qs 100 |

a.m. = active material

Composition (B1)

| Ingredients | Amount (g) |
|---|---|
| Amine compound 1, ingredient iv) | 50 (a.m.) |
| Isododecane, ingredient iii) | 50 |

After application of compositions (A1) and (B1), deposits that are not tacky (++), transfer-resistant and resistant to daily attacking factors such as food oil (olive oil) and water (++) are finally observed, whereas when composition (B1) is not applied, the tackiness is very much present and unsatisfactory (−−).

### Example 2: Formula Containing Only Volatile Oils—Polymer of Example 1+Amine Compound 2

Composition (A2)

| Ingredients | Amount (g) |
|---|---|
| Combination of particle example 1, ingredients i), ii) | 25 (a.m.) |
| Pigment paste 40 wt % of DC Red 7 in isododecane, | 5 |
| ingredient v) | |
| Isododecane, ingredient iii) | qs 100 |

Composition (B2.

| Ingredients | Amount (g) |
|---|---|
| Amine compound 2, ingredient iv) | 10 (a.m.) |
| Isododecane, ingredient iii) | 90 |

After application of compositions (A2) and (B2), deposits that are not tacky (++), transfer-resistant (++) and resistant to daily attacking factors such as food oil (olive oil) and water (++) are finally observed, whereas when composition (B2) is not applied, the tackiness is very much present and unsatisfactory (−−).

### Example 3: Formula Containing Only Volatile Oils—Polymer of Example 2+Amine Compound 1

Composition (A3)

| Ingredients | Amount (g) |
|---|---|
| Combination of particle example 2, ingredients i), ii) | 25 (a.m.) |
| Pigment paste 40 wt % of DC Red 7 in isododecane, | 5 |
| ingredient v) | |
| Isododecane, ingredient iii) | qs 100 |

Compostion (B3)

| Ingredients | Amount (g) |
|---|---|
| Amine compound 1, ingredient iv) | 50 (a.m.) |
| Isododecane, ingredient iii) | 50 |

After application of compositions (A3) and (B3), deposits that are not tacky (++), transfer-resistant (++) and resistant to daily attacking factors such as food oil (olive oil) and water (++) are finally observed, whereas when composition (B3) is not applied, the tackiness is very much present and unsatisfactory (−−) and the resistance to oil is less correct (−).

### Example 4: Formula Containing Nonvolatile Oils: Polymer of Example 1+Amine Compound 1−Nonvolatile Oil: Pentaphenyl Silicone DC 555

Composition (A4)

| Ingredients | Amount (g) |
|---|---|
| Combination of particle example 1, ingredients i), ii) | 20 (a.m.) |
| Pigment paste 40 wt % of DC Red 7 in isododecane, | 5 |
| ingredient v) | |
| DC 555 ingredient iii) | 20 |
| Isododecane, ingredient iii) | qs 100 |

Composition (B4)

| Ingredients | Amount (g) |
|---|---|
| Amine compound 1, ingredient iv) | 50 (a.m.) |
| Isododecane, ingredient iii) | 50 |

After application of compositions (A4) and (B4), deposits that are not tacky (++), transfer-resistant (++) and resistant to daily attacking factors such as food oil (olive oil) and water (++) are finally observed, whereas when composition (B4) is not applied, the tackiness is very much present and unsatisfactory (−−) and the resistance to oil is not satisfactory either (−−).

### Example 5: Formula Containing Nonvolatile Oils: Polymer of Example 2+Amine Compound 1−Nonvolatile Oil: Pentaphenyl Silicone DC 555

Composition (A5)

| Ingredients | Amount (g) |
|---|---|
| Combination of particle example 2, ingredients i), ii) | 20 (a.m.) |
| Pigment paste 40 wt % of DC Red 7 in isododecane, | 5 |
| ingredient v) | |
| DC 555 ingredient iii) | 20 |
| Isododecane, ingredient iii) | qs |

Composition (B5)

| Ingredients | Amount (g) |
|---|---|
| Amine compound 1, ingredient iv) | 50 (a.m.) |
| Isododecane, ingredient iii) | 50 |

After application of compositions (A5) and (B5), deposits that are not tacky (++), transfer-resistant (++) and resistant to daily attacking factors such as food oil (olive oil) and water (++) are finally observed, whereas when composition (B5) is not applied, the tackiness is very much present and unsatisfactory (−−) and the resistance to oil is not satisfactory either (−−).

### Example 6: Formula Containing Nonvolatile Oils: Polymer of Example 1+Amine Compound 1−Nonvolatile Oils: Octyldodecanol and Isododecane

Composition (A6)

| Ingredients | Amount (g) |
|---|---|
| Combination of particle example 1, ingredients i), ii) | 20 (a.m.) |
| Pigmentary paste 40 wt % of DC Red 7 in isododecane, | 5 |
| ingredient v) | |
| Octyldodecanol, ingredient iii) | 20 |
| Isododecane, ingredient iii) | qs |

Composition (B6)

| Ingredients | Amount (g) |
|---|---|
| Amine compound 1, ingredient iv) | 50 (a.m.) |
| Isododecane, ingredient iii) | 50 |

After application of compositions (A6) and (B6), deposits that are not tacky (++), transfer-resistant (++) and resistant to daily attacking factors such as food oil (olive oil) and water (++) are finally observed, whereas when composition (B6) is not applied, the tackiness is very much present (−−) and unsatisfactory and the resistance to oil is not satisfactory either (−−).

1. A process for treating keratin materials comprising:
1) the application to said materials of an oily dispersion (A) comprising:
i) one or more particles consisting of one or more ethylenic copolymers:
a) of (C₁-C₄)alkyl (C₁-C₄)(alkyl)acrylate; and
b) of ethylenically unsaturated anhydride compound; and
ii) one or more stabilizers consisting of ethylenic polymers chosen from:
c) polymers of (C₃-C₁₂)cycloalkyl acrylate or (C₁-C₆)(alkyl)acrylate monomers; and
d) copolymers of (C₃-C₁₂)cycloalkyl acrylate or (C₁-C₆)(alkyl)acrylate and (C₁-C₄)alkyl (C₁-C₄)(alkyl)acrylate; and
iii) one or more hydrocarbon-based oils;
2) the application to said materials of a composition (B) comprising:
iv) one or more amine compound(s) chosen from:
e) polyamine compounds bearing several primary amine and/or secondary amine groups, and
f) amino alkoxysilanes,
it being understood that:
the process uses v) one or more cosmetic active agents chosen from a) pigments, b) active agents for caring for keratin materials and c) UV-screening agents, and also d) mixtures thereof, ingredients a) to d) possibly being found in composition (A), and/or in composition (B), and/or in another composition (C); and
compositions (A), (B) and (C) may be applied together or separately.

2. A process for treating keratin materials according to claim 1, comprising:
1) the application to said materials of an oily dispersion (A), comprising:
i) one or more particles consisting of one or more ethylenic copolymers:
a) of (C₁-C₄)alkyl (C₁-C₄)(alkyl)acrylate; and
b) of ethylenically unsaturated anhydride compound; and
ii) one or more stabilizers consisting of ethylenic polymers chosen from:
c) polymers of (C₃-C₁₂)cycloalkyl acrylate or (C₁-C₆)(alkyl)acrylate monomers; and
d) copolymers of (C₃-C₁₂)cycloalkyl acrylate or (C₁-C₆)(alkyl)acrylate and (C₁-C₄)alkyl (C₁-C₄)(alkyl)acrylate; and
iii) one or more hydrocarbon-based oil(s).

3. The process as claimed in claim 1 in which the oily dispersion (A) comprises one or more particles constituted of one or more ethylenic copolymers a) of (C₁-C₄)alkyl (C₁-C₄)(alkyl)acrylate, of formula H₂C=C(R)—C(O)—O—R′, with R representing a hydrogen atom or a (C₁-C₄)alkyl group, and R¹ representing a linear or branched (C₁-C₄)alkyl.

4. The process as claimed in claim 1, in which the oily dispersion (A) comprises one or more particles constituted of one or more ethylenic copolymers of b) ethylenically unsaturated anhydride compound b) chosen from derivatives of maleic anhydride (Ib) and of itaconic anhydride (IIb): 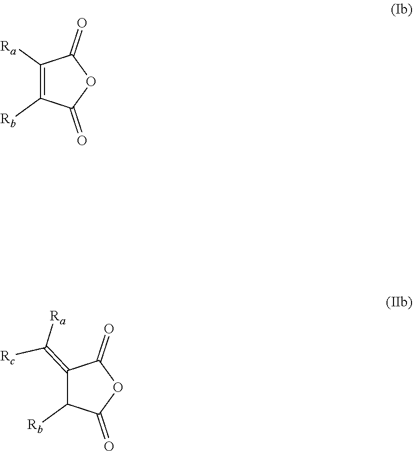
in which formulae (Ib) and (IIb) Rₐ, R_{b} and R_{c}, which may be identical or different, represent a hydrogen atom or a (C₁-C₄)alkyl group.

5. The process as claimed in claim 1, in which the polymer(s) of the particles i) of dispersion (A) comprise, or consist essentially of, from 80% to 99.99% by weight of ingredient a) relative to the total weight of the polymer(s) and from 0.01% to 20% by weight of ingredient b), relative to the total weight of the polymer.

6. The process as claimed in claim 1, in which the copolymer(s) of the particles i) consist of polymers a) and b) with an a)/b) weight ratio inclusively between 5.5 and 20.

7. The process as claimed in claim 1, in which the polymer of the particles i) is present in dispersion (A) in an amount ranging from 20% to 60% by weight relative to the total weight of dispersion (A).

8. The process as claimed in claim 1, in which the stabilizer(s) ii) of dispersion (A) are constituted of ethylenic polymers chosen from:
c) polymers of monomers of formula H₂C=C(R)—C(O)—O—R″ with R representing a hydrogen atom or (C₁-C₄)alkyl group, and R″ representing a (C₅-C₁₀)cycloalkyl group; and
d) copolymers of H₂C=C(R)—C(O)—O—R′ and of H₂C=C(R)—C(O)—O—R″ with R, R′ and R″ as defined previously.

9. The process as claimed in claim 1, in which the sum of i) the particle(s)+ii) stabilizer(s) present in dispersion (A) comprises from 10% to 50% by weight of copolymers d and from 50% to 90% by weight of polymer c) relative to the total weight of the sum of i) stabilizer(s) and ii) of polymer particle(s).

10. The process as claimed in claim 1, in which the hydrocarbon-based oil(s) iii) of dispersion (A) are apolar, i.e. formed solely of carbon and hydrogen atoms.

11. The process as claimed in claim 1, in which dispersion (A) is in inverse emulsion, i.e. of water-in-oil (W/O) type, and comprises one or more surfactants.

12. The process as claimed in claim 1, in which the amine compound(s) of composition (B) are chosen from the amino alkoxysilanes f).

13. The process as claimed in claim 1, in which the a compound(s) of composition (B) are chosen from e) polyamine compounds bearing several primary amine and/or secondary amine groups

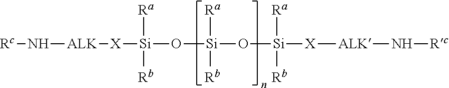

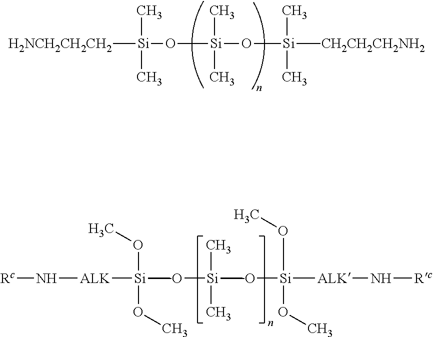

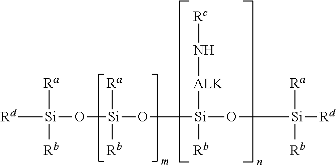

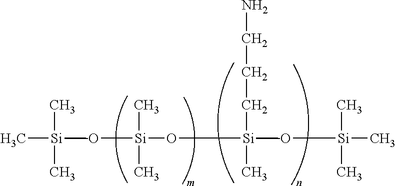

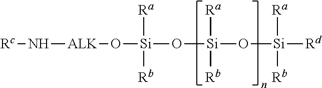

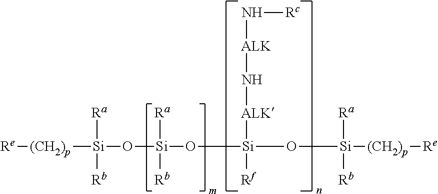

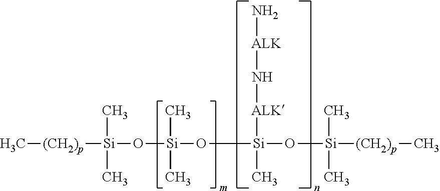

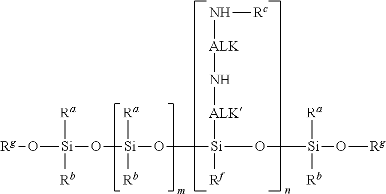

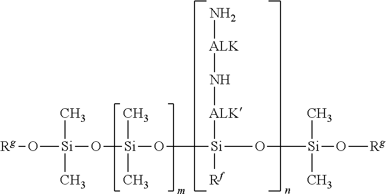

14. The process as claimed in claim 1, in which composition (B) comprises one or more hydrocarbon-based oil(s) iii).

15. The process as claimed in claim 1, in which composition (B) is aqueous.

16. The process as claimed in claim 1, in which composition (B) is anhydrous.

17. The process as claimed in claim 1, in which v) the cosmetic agent(s) are chosen from pigments.

18. The process as claimed in claim 1, in which v) the cosmetic agent(s) are in dispersion (A).

19. The process as claimed in claim 1, in which v) the cosmetic active agent(s) are in composition (B).

20. The process as claimed in claim 1, which uses one or more plasticizers vi), which are present in composition (A), and/or in composition (B), and/or in another composition (C).

21. The process as claimed in claim 1, comprising:
1) the application to said materials of an oily dispersion (A); followed by
2) the application to said materials of a composition (C) comprising v) one or more cosmetic active agents, preferably pigments; followed by
3) the application to said materials of a composition (B).

22. The process as claimed in claim 1, comprising:
1) the application to said materials of a composition (C) comprising v) one or more cosmetic active agents; followed by
2) the application to said materials of an oily dispersion (A); followed by
3) the application to said materials of a composition (B).

23. The process as claimed in claim 1, in which, between step 1) and step 2), there is no rinsing, and the keratin materials are dried naturally before applying composition (B).

24. A kit or device with several separate compartments, comprising:
in a first compartment: an oily dispersion (A) comprising:
i) one or more particles consisting of one or more ethylenic copolymers:
a) of (C₁-C₄)alkyl (C₁-C₄)(alkyl)acrylate; and
b) of ethylenically unsaturated anhydride compound; and
ii) one or more stabilizers consisting of ethylenic polymers chosen from:
c) polymers of (C₃-C₁₂)cycloalkyl acrylate or (C₁-C₆)(alkyl)acrylate monomers; and
d) copolymers of (C₃-C₁₂)cycloalkyl acrylate or (C₁-C₆)(alkyl)acrylate and (C₁-C₄)alkyl (C₁-C₄)(alkyl)acrylate; and
iii) one or more hydrocarbon-based oils:
in a second, separate compartment: a composition (B) comprising:
iv) one or more amine compound(s) chosen from:
e) polyamine compounds bearing several primary amine and/or secondary amine groups, and
f) amino alkoxysilanes, and
optionally in a third compartment separate from the other two: a composition (C) comprising one or more cosmetic agents chosen from a) pigments, b) active agents for caring for keratin materials and e) UV-screening agents, and also d) mixtures thereof.

25. The oily dispersion (A) as defined in claim 1, comprising v) one or more cosmetic active agents it being understood that, when v) represents one or more pigment(s), then the dispersion is anhydrous and does not comprise any polyamine compound bearing several primary amine and/or secondary amine groups and does not comprise any amino alkoxysilanes.

26. An oily dispersion (A) comprising:
i) one or more particles consisting of one or more ethylenic copolymers:
a) of (C₁-C₄)alkyl (C₁-C₄)(alkyl)acrylate; and
b) of ethylenically unsaturated anhydride compound; and
ii) one or more stabilizers consisting of ethylenic polymers chosen from:
c) polymers of (C₃-C₁₂)cycloalkyl acrylate or (C₁-C₆)(alkyl)acrylate monomers; and
d) copolymers of (C₃-C₁₂)cycloalkyl acrylate or (C₁-C₆)(alkyl)acrylate and (C₁-C₄)alkyl (C₁-C₄)(alkyl)acrylate; and
iii) one or more hydrocarbon-based oils, and wherein the dispersion (A) is in inverse emulsion, i.e. of water-in-oil (W/O) type, and comprises one or more surfactants.
